(19) United States
(12) Patent Application Publication
(10) Pub. No.: US 2025/0218533 A1
(43) Pub. Date: Jul. 3, 2025

(54) METHODS FOR DETECTING FETAL COPY NUMBER VARIATION THROUGH NON-INVASIVE PRENATAL TESTING

(71) Applicant: HOAI NGHIA NGUYEN, Ho Chi Minh (VN)

(72) Inventors: HOAI NGHIA NGUYEN, Ho Chi Minh (VN); HOA GIANG, Ho Chi Minh (VN); THI TUONG VY NGUYEN, Ho Chi Minh (VN); MINH DUY PHAN, Brisbane (AU); VINH NGUYEN, Ho Chi Minh (VN)

(73) Assignee: GENE SOLUTIONS JOINT STOCK COMPANY, Ho Chi Minh (VN)

(21) Appl. No.: 19/002,783

(22) Filed: Dec. 27, 2024

Related U.S. Application Data
(60) Provisional application No. 63/615,773, filed on Dec. 28, 2023.

(51) Int. Cl. G16B 20/10; C12Q 1/6806; G16B 30/10; G16B 40/20; G16H 50/30

(52) U.S. Cl. CPC G16B 20/10; C12Q 1/6806; G16B 30/10; G16B 40/20; G16H 50/30

(57) ABSTRACT

A method for detecting fetal CNVs through non-invasive prenatal testing, comprising steps: (a) collecting blood samples from pregnant women; (b) extracting cfDNA fragments from the blood samples, performing whole genome sequencing on extracted cfDNA fragments to obtain cfDNA sequencing data, and preprocessing cfDNA sequencing data by removing adapters, aligning and mapping reads to a referenced human genome; (c) performing quality control on obtained cfDNA sequencing data for being accepted for the prediction; (d) dividing referenced genome into a plurality of non-overlapping bins and filtering the bins based on a predetermined GC-content threshold for bins; (e) defining a CNV detection window, a bin size, a set of features for machine learning/deep learning models and fine-tune model for detecting fetal CNV for selecting a final model; and (f) applying the final model to predict fetal CNVs, including microdeletion syndromes, or microduplications, or aneuploidies, or the number of sex chromosomes.

### CLAIM OF PRIORITY

This application claims the benefit of U.S. Provisional Application Ser. No. 63/615,773, entitled “Methods for detecting embryonic copy number variation through non-invasive prenatal testing”, filed on Dec. 28, 2023. The patent application identified above is incorporated here by reference in its entirety to provide continuity of disclosure.

### FIELD OF THE INVENTION

The present invention relates to the field of biological information, specifically non-invasive prenatal testing (NIPT). In particular, the invention relates to the detection of fetal chromosomal abnormalities, and more particularly of fetal aneuploidies. More specifically, the present invention relates to methods for detecting fetal copy number variation (CNV) through non-invasive prenatal testing.

### BACKGROUND ART

One of the critical endeavors in biomedical research is the identification and characterization of genetic aberrations associated with adverse health outcomes. Numerous studies have elucidated specific genes and diagnostic markers within genomic regions exhibiting abnormal copy number variations (CNVs). These CNVs have been implicated in various pathological conditions. In the field of prenatal diagnostics, for example, the presence of additional or missing copies of certain chromosomal segments has been linked to significant congenital issues. Such genetic anomalies can result in lifelong medical complications and potentially reduced life expectancy for affected individuals.

CNV represents a major structural variation in the genome, encompassing duplications and deletions of chromosomal segments typically ranging from 1 kilobase to 20 megabases in length. These genomic alterations have been associated with a variety of genetic disorders and neurodevelopmental conditions, such as autism spectrum disorders, schizophrenia, intellectual disability, developmental delays, and various congenital anomalies.

Non-invasive prenatal testing (NIPT) is an advanced prenatal screening technology used during pregnancy. It leverages the presence of cell-free fetal DNA circulating in maternal peripheral blood. NIPT offers high detection accuracy while eliminating the risks of miscarriage and intrauterine infection associated with invasive procedures such as chorionic villus sampling, amniocentesis, and transabdominal venipuncture.

The principle underlying NIPT involves extracting cell-free DNA (cfDNA) from maternal plasma encompassing cfDNA originating from both the mother and the fetus, constructing a next-generation sequencing (NGS) library, and employing NGS technology to analyze the sequence information of the maternal plasma cfDNA, thereby detecting aberration in the fetal cell-free DNA. However, current NIPT methods face limitations, including reduced sensitivity due to the low levels of fetal cfDNA and the size of genomic aberrations (ie. chromosomal anomalies versus CNVs). These limitations highlight the need for improved non-invasive methods that offer enhanced specificity, sensitivity, and applicability for reliably diagnosing CNVs in diverse clinical settings.

Many inventions in NIPT (e.g., WO2023031641, US20240013859A1, and U.S. Pat. No. 11,437,121B2) have addressed batch effects caused by experiment analysis operators, time, platform, and laboratory environment. These batch effects significantly impact analysis results, potentially leading to false negative, false positive, or undetermined outcomes. These outcomes necessitate data re-verification, increasing the testing cost and duration. To mitigate these issues, various correction methods have been proposed to partially eliminate the technical variance related to GC-content, mappability, and sequencing errors.

Some inventions rely on Z-score calculation or certain transformations of Z-score to detect the abnormal signals relative to the normal signals from a set of healthy samples. For instance, patent application No. WO2023031641 describes a method to detect abnormalities by inspecting signals at gene segments on X and Y chromosomes to identify the number of fetal sex chromosomes. This application further employs Z-score calculation to identify fetal aneuploidies on chromosomes 13, 18, and 21. Similarly, application No. KR20210130680A utilizes a two-layer Z-score transformation to detect aneuploidies. However, all Z-score strategies depend on the selection of healthy samples as reference, which is susceptible to the low amount and highly variable fetal fractions (the amount of fetal cfDNA in the total cfDNA obtained from pregnant woman) across samples.

Phan et al. (2018) identified trisomies through Differential Proportion (DP) calculation, which amplifies the relative differences between fetal and maternal signals in the sample while eliminating the need for technical variance correction across all chromosomal regions. This work inspired and formed a foundation for methods dissecting intra-sample differences required to detect fetal CNVs.

The invention described in U.S. application Ser. No. 12/100,483B2 proposes a screening method based on targeted sequencing, a cost-effective approach in which the hybridization probes were used to target specific regions in the chromosome known to exhibit CNVs. However, this method introduces uneven sequencing coverage, posing a significant challenge for CNV detection. Furthermore, the targeted sequencing approach limits the number of CNV types that can be identified from a single blood draw, thereby hindering the expansion of NIPT to all chromosome segments. These limitations motivate the development of high-resolution CNV detection methods applicable to any genomic segment.

A general and expandable method for determining fetal genetic abnormalities through cfDNA is still lacking. This method should be able to detect all types of copy number changes, encompassing all chromosomal aneuploidy plus all CNVs, through a uniform approach.

To enable the detection of all fetal CNVs via maternal plasma cfDNA, the method should focus on identifying the difference in copy number among genomic segments within one sample (ie. intra-difference). This approach will eliminate the dependence on referenced samples, potentially leading to more robust and accurate fetal CNV detection.

Finally, it is necessary to develop a method to build and train models that:
a) Provide a reference-free model (ie. detecting intra-difference) to detect fetal aneuploidies in all autosomes with comparable or better performance and reliability than existing approaches for non-invasive prenatal testing;
b) Enable prediction of (i) fetal CNVs, including microdeletions such as DiGeorge syndrome, Wolf-Hirschhorn syndrome, Cri-du-chat syndrome, Prader-Willis/Angelman syndrome, microduplication of 22q11.2, (ii) abnormal number of sex chromosomes, and (iii) a broader selection of other disorders during pregnancy; and
c) Remain cost-effective and non-invasive without compromising diagnostic accuracy during pregnancy.

The invention provides solutions to achieve the above objectives.

### SUMMARY OF THE INVENTION

The invention team of this application broke through the limitations of traditional detection methods and developed a set of techniques for detecting CNV from cell-free DNA in pregnant women's blood samples. This technique plays an important role in solving problems such as fetal genetic abnormality screening.

Accordingly, an objective of the present invention is to provide a method for detecting fetal CNV through non-invasive prenatal testing (NIPT), comprising steps performed in the following specific order:
(a) collecting blood samples from pregnant women from 9 weeks of pregnancy;
(b) extracting cell-free deoxyribonucleic acid fragments (cfDNA fragments) from the blood samples, performing whole genome sequencing (WGS) on said extracted cfDNA fragments to obtain cfDNA sequencing data, and preprocessing cfDNA sequencing data by removing adapters, aligning and mapping reads to a referenced human genome;
(c) performing quality control on obtained cfDNA sequencing data for being accepted for the prediction based on the total number of reads, the total number of reads successfully mapped relative to the total number of reads sequenced, the average sequencing coverage or average sequencing depth, and the read duplication rate;
(d) dividing said referenced genome into a plurality of non-overlapping bins and filtering the bins based on a predetermined GC-content threshold for bins;
(e) defining a CNV detection window, a bin size, a set of features for machine learning/deep learning models and fine-tune said model for detecting fetal CNV for selecting a final model; and
(f) applying the final model to predict CNVs, including microdeletion syndromes, or microduplications, or aneuploidies, or the number of sex chromosomes, using at least three parameters, including the size of CDW, the level of significance of RCᵢ and/or DPᵢ different from the window mean, and the set of features chosen from quantitative signals.

Another objective of the present invention is to provide the set of features for model learning by transforming the raw read counts of kept bins into a set of quantitative copy number signals at bins of interest, then using these quantitative signals as features for a model, allowing the model to learn from the features to detect CNV; in which the features is selected from the set of quantitative signals (QS), including (A) relative counts (RCᵢ with i among the plurality of bins), (B) difference in proportion (DPᵢ with i among the plurality of bins), (C) relative differences of RCᵢ or DPᵢ and the mean RC or DP across all bins in the CDW, and (D) percentages of read counts of chromosomes of interest in the sample of interest over the total number of read counts in the sample of interest perc_{ch}_{ₖ};
(A) RCᵢ is defined as:

$${RC}_{i} = \frac{fetal read {counts}_{i}}{total read counts of sample}$$

(B) DPᵢ is defined as:

$${DP}_{i} = \frac{fetal read {counts}_{i}}{total fetal read counts of sample} - \frac{maternal read {counts}_{i}}{total maternal read counts of sample}$$

wherein the fetal read counts correspond to the fetal fragment size, in which the fetal fragment size is below 140 base-pairs, below 145 base-pairs, below 150 base-pairs, below 155 base-pairs, below 160 base-pairs, below 160 base-pairs, or below 170 base-pairs;
wherein the maternal read counts correspond to the maternal fragment size, in which the maternal fragment size is above 160 base-pairs, above 165 base-pairs, above 170 base-pairs, and above 175 base-pairs;
(C) the relative difference between the quantitative signal RCᵢ or DPᵢ and the mean RC or DP of all bins across the CDW encoded in binaries (0 and 1) or encoded in levels of significant difference α;
   wherein the relative difference between the quantitative signal RCᵢ or DPᵢ and the mean RC or DP of all bins across the CDW encoded in binaries (0 and 1);

$${QS}_{i} = \{ \begin{matrix}1 ( deletion ) & if {QS}_{i} \leq mean ( {\sum }_{i = 1}^{n} {QS}_{i} ) - α \times \sqrt{\frac{{\sum }_{i = 1}^{n} {( {QS}_{i} - mean ( {\sum }_{i = 1}^{n} QS ) )}^{2}}{n}} \\ 1 ( duplication ) & if {QS}_{i} \geq mean ( {\sum }_{i = 1}^{n} {QS}_{i} ) + α \times \sqrt{\frac{{\sum }_{i = 1}^{n} {( {QS}_{i} - mean ( {\sum }_{i = 1}^{n} QS ) )}^{2}}{n}} \\ 0 ( normal ) , & otherwise\end{matrix}$$

QSᵢ is the quantitative signal RCᵢ or DPᵢ at bin i;
α ranging between 0.5-2 indicates the significance of the relative difference between QSᵢ and mean QS of all bins across the CDW; and
n is the total number of bins of interest in the CDW;
wherein the relative difference between the quantitative signal RCᵢ or DPᵢ and the mean RC or DP of all bins across the CDW encoded in levels of significant difference α;

$${QS}_{i}^{′} = \{ \begin{matrix}α + b ( deletion ) & if {QS}_{i} \leq mean ( {\sum }_{i = 1}^{n} {QS}_{i} ) - α \times \sqrt{\frac{{\sum }_{i = 1}^{n} {( {QS}_{i} - mean ( {\sum }_{i = 1}^{n} QS ) )}^{2}}{n}} \\ α + b ( duplication ) & if {QS}_{i} \geq mean ( {\sum }_{i = 1}^{n} {QS}_{i} ) + α \times \sqrt{\frac{{\sum }_{i = 1}^{n} {( {QS}_{i} - mean ( {\sum }_{i = 1}^{n} QS ) )}^{2}}{n}} \\ 0 ( normal ) , & otherwise\end{matrix}$$

QSᵢ is the quantitative signal RCᵢ or DPᵢ at bin i;
QS′ᵢ is the new quantitative signal obtained from QSᵢ;
α ranging between 0.5-2 indicates the significance of the relative difference between QSᵢ and mean QS of all bins across the CDW,
b=0.5 if α ranging between 0.5 and 1.5, b=1 if α ranging between 0.5 and 2; and
n is the total number of bins of interest in the CDW;
(D) perc_{chr}_{ₖ} is defined as:

$${perc}_{{chr}_{k}} = \frac{{RPM}_{{chr}_{k}}}{Total RPM} \times 100 with {RPM}_{{chr}_{k}} = \frac{\frac{read {counts}_{{chr}_{k}}}{{l}_{{chr}_{k}}}}{{\sum }_{j = 1}^{23} \frac{read {counts}_{{chr}_{k}}}{{l}_{{chr}_{k}}}}$$

l is the length of chromosome of interest;
RPM_{chr}_{ₖ} is the normalized read counts of chromosome of interest in the sample of interest; and
Total RPM is the total normalized number of read counts in the sample of interest.

It is moreover the objective of the invention to provide the set of parameters is determined for a specific CNV is determined by fine-tuning models for detecting fetal copy number variation by performing the steps in the following order:
(i) one by one through each defined CDW for each CNV, for which one can apply a strategy of sliding window of at least 1 Mb with seeding length of four consecutive bins;
(ii) calculating the relative differences of RCᵢ and DPᵢ at each bin i in the CDW, combined with features from RCᵢ, DPᵢ and perc_{chr}_{ₖ}, which is calculated independently from said chosen CDW;
(iii) choosing levels of significant α for RCᵢ and DPᵢ by comparing RCᵢ and/or DPᵢ at each bin at step (ii) to the mean of all RCᵢ and/or DPᵢ in the CDW;
(iv) one by one choosing any set of features from the plurality of quantitative signals including RCᵢ, DPᵢ, relative differences of RCᵢ and/or DPᵢ compared to mean RCᵢ and/or DPᵢ across all bins of the CDW, and perc_{chr}_{ₖ} for the learning model;
(v) one by one applying any learning model on the training set, with three main parameters including the size of CDW at step (i), the level of significance α or binaries encoding of RCᵢ and/or DPᵢ determined at step (iii), and the set of features chosen from quantitative signals at step (iv), and with the hyperparameters of the learning model defined during the training process;
   wherein the learning model is selected from the group consisting of machine learning-based models, Gaussian Mixture Model (GMM), and Hidden Markov Model;
   wherein machine learning-based models include Naïve Bayes, K-Nearest Neighbors, Random Forest, Multi-Layer Perceptron, Support Vector Machines, or any other machine learning-based models;
(vi) applying the trained model at step (v) to predict the CNV on the test set and evaluating if the model achieves the targeted performance metrics;
   wherein the targeted performance metrics include:
   area under the curve (AUC) is from 0.9 and above;
   accuracy is from 0.9 and above;
   sensitivity is from 0.9 and above;
   specificity is from 0.95 and above; and
   positive predictive value is from 0.75 and above;
   mean squared error (MSE) is below 0.2;
(vii) Rank the models that satisfy the targeted performance metrics and choose the best performance model as the final model for the CNV of interest; if no model is found to satisfy the targeted performance metrics, steps (iii) to (vii), or steps (iv) to (vii), or steps (v) to (vii), or steps (i) to (vii) will be repeated until a model is found.

Finally, another objective of the present invention is to provide a system for evaluation of fetal CNV in a test sample through non-invasive prenatal testing (NIPT), the system comprising:
a sequencer for receiving cell-free deoxyribonucleic acid fragments (cfDNA fragments) from the test sample and providing cfDNA sequencing data of the test sample;
a computer; and
one or more computer-readable storage media having stored thereon instructions for execution on said computer to:
   (a) collecting blood samples from pregnant women from 9 weeks of pregnancy;
   (b) extracting cfDNA fragments from the blood samples, performing whole genome sequencing on said extracted cfDNA fragments to obtain cfDNA sequencing data, and preprocessing cfDNA sequencing data by removing adapters, aligning and mapping reads to a referenced human genome;
   (c) performing quality control on obtained cfDNA sequencing data for being accepted for the prediction;
   (d) dividing said referenced genome into a plurality of non-overlapping bins and filtering the bins based on a predetermined GC-content threshold for bins;
   (e) defining a CNV detection window, a bin size, a set of features for machine learning/deep learning models and fine-tune said model for detecting fetal CNV for selecting a final model; and
   (f) applying the final model to predict CNVs, including microdeletion syndromes, or microduplications, or aneuploidies, or the number of sex chromosomes, using at least three parameters, including the size of CDW, the level of significance α for RCᵢ and/or DPᵢ, and the set of features chosen from quantitative signals.

These and other advantages of the present invention will no doubt become obvious to those of ordinary skill in the art after having read the following detailed description of the preferred embodiments, which are illustrated in the various drawing Figures.

### BRIEF DESCRIPTION OF THE DRAWINGS

The accompanying drawings, which are incorporated in and form a part of this specification, illustrate embodiments of the invention and, together with the description, explain the principles of the invention.

FIG. 1 is a conceptual block diagram illustrating the principle of selecting a final model to predict the CNVs possibility of microdeletion syndromes, or microduplications, or aneuploidies, or the number of sex chromosomes in accordance with an exemplary embodiment of the present invention;

FIG. 2 is a flowchart illustrated the method for detecting fetal CNV through NIPT by applying the final model to predict the CNVs on new samples including microdeletion syndromes, or microduplications, or aneuploidies, or the number of sex chromosomes according to embodiment of the present invention;

FIG. 3a is a graph illustrated the training dataset before data point generation using the distribution estimated by GMM according to embodiment of the present invention;

FIG. 3b is a graph illustrated the training dataset after data point generation using the distribution estimated by GMM according to embodiment of the present invention;

FIG. 4a is a graph illustrated the training dataset before PCA correction, wherein CNV-free data points (yellow) containing unwanted variation are located far from other CNV-free data points (green), according to embodiment of the present invention; and

FIG. 4b is a graph illustrated the training dataset after PCA correction, wherein CNV-free data points (yellow) containing unwanted variation are located among other CNV-free data points (green), according to embodiment of the present invention.

### DETAILED DESCRIPTION OF THE INVENTION

Reference will now be made in detail to the preferred embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications, and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of ordinary skill in the art that the present invention may be practiced without these specific details. In other instances, well-known methods, procedures, components, and circuits have not been described in detail so as not to unnecessarily obscure aspects of the present invention.

It should be noted that the terms “comprises” and “comprising”, and “the” and “these” are intended to cover a non-exclusive inclusion, for example, a process, method, system, product, or device that comprises a series of steps or units is not necessarily limited to those steps or units may include other steps or units not explicitly listed or inherent to such processes, methods, products or devices.

The headings provided herein are not intended to limit the disclosure.

In the following, in order to facilitate the understanding of the present solution, some proper nouns appearing in the following embodiments of the present application are explained:
the term “cfDNA” refers to extracellular DNA fragments circulating in body fluids, primarily blood plasma, that are not enclosed within cellular membranes. In the context of prenatal testing, cfDNA from a pregnant woman's blood comprises maternal cfDNA and fetal cfDNA, the latter of which can be analyzed for fetal genetic abnormalities. The term “cfDNA” includes, but is not limited to, circulating cell free DNA, or cell-free DNA (cfDNA) for short. cfDNA is known to originate from various cellular processes, including apoptosis, necrosis, and active secretion, and can be derived from both normal and pathological cells. In the context of the present invention, the cfDNA of interest originates from fetal DNA and is circulating in the maternal blood. cfDNA is fragmented differently depending on its origin and is a small part of the genome from which it originates. Therefore, fetal cfDNA carries various characteristics that set it apart from the cfDNA derived from maternal cells;
the term “GC content”: the proportion of guanine (G) and cytosine (C) nucleotides relative to the total number of nucleotides in a specified region of interest on a human chromosome is the GC content of the region of interest, expressed as ratio or percentage;
the term “fastq”: a standardized file formant used in high-throughput sequencing for storing raw sequencing data, including both sequence information and corresponding quality scores;
the terms “aligned”, “alignment”, or “aligning” refer to the computational process of comparing a sequencing read to a referenced genome to determine the potential location on the referenced genome where the read is similar or identical to. The location on the referenced genome where the read is similar to is the mapping location, ie. the read is mapped or aligned to a particular location in the referenced genome. In some cases, alignment simply used to indicate whether a read is present in a particular referenced sequence (i.e., whether the read is derived from the referenced sequence). For example, the alignment of a read to the referenced sequence for human chromosome 13 will tell whether the read is present in the human chromosome 13. In some cases, an alignment additionally indicates a location in the referenced sequence where the read is mapped to. For example, if the referenced sequence is the whole human genome sequence, an alignment may indicate that a read is of chromosome 13, and may further indicate that the read is of a particular arm or locus of chromosome 13;
the term “mapping” as used herein refers to the specific assignment of sequence reads to a larger sequence, such as a referenced genome, by alignment;
the term “paired-end read” refers to a pair of reads obtained from paired-end sequencing, in which each read from the same pair originates from each end of the same DNA fragment;
the term “sensitivity” as used herein refers to the probability of a test correctly identifying individuals with the condition of interest. It is calculated as the ratio of true positive results to the sum of true positive and false negative results, expressed as a percentage;
the term “specificity” as used herein denotes the probability of a test correctly identifying individuals without the condition of interest. It is calculated as the ratio of true negative results to the sum of true negative and false positive results, expressed as a percentage; and
In this article, the term “based on” when used in the context of obtaining a specific quantitative value, refers to using another quantity as an input to calculate as a specific quantitative value as an output.

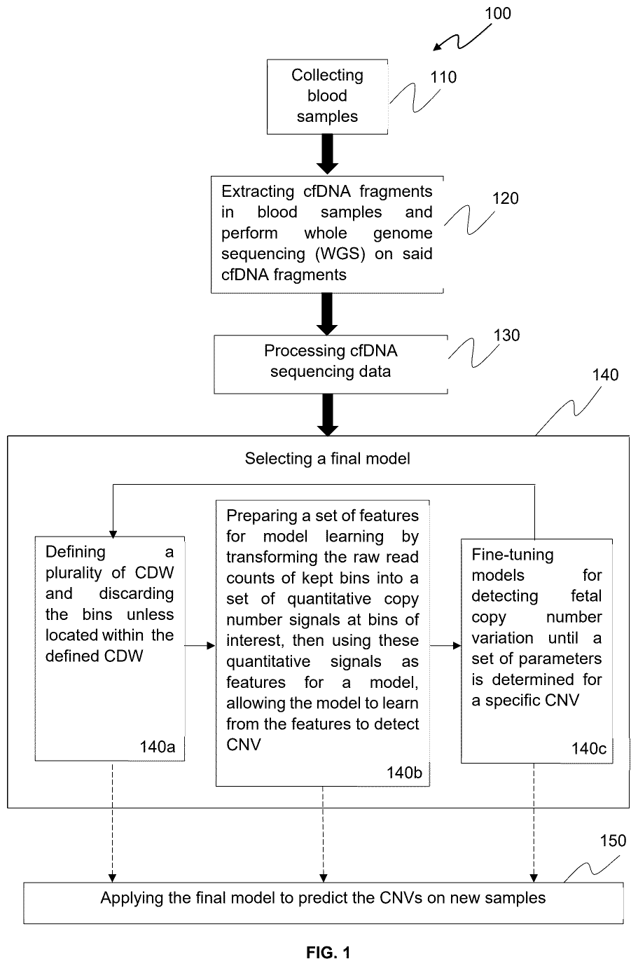

One embodiment of the invention is now described with reference to FIG. 1. FIG. 1 is a conceptual block diagram illustrating the principle of selecting a final model to predict the CNVs possibility of microdeletion syndromes, or microduplications, or aneuploidies, or the number of sex chromosomes 100 (“method 100”) in accordance with an exemplary embodiment of the present invention.

At step 110, collecting blood samples from pregnant women from 9 weeks of pregnancy.

At step 120, extracting cell-free deoxyribonucleic acid fragments (cfDNA fragments) from the blood samples, performing whole genome sequencing (WGS) on said extracted cfDNA fragments to obtain cfDNA sequencing data, and preprocessing cfDNA sequencing data by removing adapters, aligning and mapping reads to a referenced human genome.

At step 130, processing cfDNA sequencing data includes performing quality control on obtained cfDNA sequencing data at step 120 for being accepted for the prediction; and dividing a referenced genome into a plurality of non-overlapping bins, and filtering the bins based on a predetermined GC-content threshold for bins.

According to the embodiment of the invention, performing quality control on obtained cfDNA sequencing data for being accepted for the prediction based on the total number of reads, the total number of reads successfully mapped relative to the total number of reads sequenced, the average sequencing coverage or average sequencing depth, and the read duplication rate;
in which the total number of reads is of at least 18M paired-end reads;
the total number of reads successfully mapped relative to the total number of reads sequenced is at least 70%;
the average sequencing coverage or average sequencing depth is between 0.1× and 5×; and
the read duplication rate is below 30%.

According to the preferred embodiment of the present invention, the average sequencing coverage (average sequencing) depth can vary from 0.1× to 1.2×.

According to the embodiment of the invention, the predetermined GC-content thresholds for bins are 30% and 70%, meaning that bins having GC-content between 30% and 70% are kept, and bins having GC-content below 30% or over 70% are removed.

According to embodiment of the present invention, the step 130 of method 100 further includes samples with the obtained cfDNA sequencing data violates one or several quality control parameters, in which the total number of reads is of at least 18M paired-end reads, the average sequencing coverage or average sequencing depth is depth below 0.1×, the total number of reads successfully mapped relative to the total number of reads sequenced is less than 70%, and the read duplication rate is more than 30%. In the present invention, the sample having outlier QC metrics are rerun for sequencing, or blood samples are noted for recollection.

At step 140, selecting a final model to predict the CNVs based on at least three parameters including the size of CNV detection window (CDW), the level of significance α or binaries encoding for RCᵢ and/or DPᵢ, and the set of features chosen from quantitative signals by repeating the steps 140a-140c until said final model achieves the targeted performance.

At step 140a, defining a plurality of CDWs, wherein a CDW is defined for each CNV type or subtype of interest, wherein a CDW encompasses a region on the genome where the CNV of interest is located.

According to embodiment of the present invention, a CDW is the region of CNV of interest, or of at least 25% bigger, or of at least 50% bigger, or of at least 75% bigger than the region of CNV of interest.

According to embodiment of the present invention, the bins are discarded unless located within the defined CDW.

At step 140b, preparing a set of features for model learning by transforming the raw read counts of kept bins at 140a into a set of quantitative copy number signals at bins of interest, then using these quantitative signals as features for a model, allowing the model to learn from the features to detect CNV;
in which the features is selected from the set of quantitative signals (QS), including (A) relative counts (RCᵢ with i among the plurality of bins), (B) difference in proportion (DPᵢ with i among the plurality of bins), (C) relative differences of RCᵢ or DPᵢ and the mean RC or DP across all bins in the CDW, and (D) percentages of read counts of chromosomes of interest in the sample of interest over the total number of read counts in the sample of interest perc_{ch}_{ₖ};
   (A) RCᵢ is defined as:

$$R {C}_{i} = \frac{fetal read {counts}_{i}}{total read counts of sample}$$

(B) DPᵢ is defined as:

$${DP}_{i} = \frac{fetal read {counts}_{i}}{total fetal read counts of sample} - \frac{maternal read {counts}_{i}}{total maternal read counts of sample}$$

wherein the fetal read counts correspond to the fetal fragment size, in which the fetal fragment size is below 140 base-pairs, below 145 base-pairs, below 150 base-pairs, below 155 base-pairs, below 160 base-pairs, below 160 base-pairs, or below 170 base-pairs;
wherein the maternal read counts correspond to the maternal fragment size, in which the maternal fragment size is above 160 base-pairs, above 165 base-pairs, above 170 base-pairs, and above 175 base-pairs;
(C) the relative difference between the quantitative signal RCᵢ or DPᵢ and the mean RC or DP of all bins across the CDW encoded in binaries (0 and 1) or encoded in levels of significant difference α;
   wherein the relative difference between the quantitative signal RCᵢ or DPᵢ and the mean RC or DP of all bins across the CDW encoded in binaries (0 and 1);

$${QS}_{i} = \{ \begin{matrix}1 ( deletion ) if Q {S}_{i} & \leq mean ( {\sum }_{i = 1}^{n} {QS}_{i} ) - α \times \sqrt{\frac{{\sum }_{i = 1}^{n} {( {QS}_{i} - mean ( {\sum }_{i = 1}^{n} QS ) )}^{2}}{n}} \\ 1 ( duplication ) if Q {S}_{i} & \geq mean ( {\sum }_{i = 1}^{n} {QS}_{i} ) + α \times \sqrt{\frac{{{\sum }_{i = 1}^{n} ( {QS}_{i} - mean {\sum }_{i = 1}^{n} QS ) )}^{2}}{n}} \\ 0 ( normal ) , otherwise & \end{matrix}$$

QSᵢ is the quantitative signal RCᵢ or DPᵢ at bin i;
α ranging between 0.5-2 indicates the significance of the relative difference between QSᵢ and mean QS of all bins across the CDW; and
n is the total number of bins of interest in the CDW;
wherein the relative difference between the quantitative signal RCᵢ or DPᵢ and the mean RC or DP of all bins across the CDW encoded in levels of significant difference α:

$${QS}_{i}^{′} = \{ \begin{matrix}α + b ( deletion ) if Q {S}_{i} & \leq mean ( {\sum }_{i = 1}^{n} {QS}_{i} ) - α \times \sqrt{\frac{{\sum }_{i = 1}^{n} {( {QS}_{i} - mean ( {\sum }_{i = 1}^{n} QS ) )}^{2}}{n}} \\ α + b ( duplication ) if Q {S}_{i} & \geq mean ( {\sum }_{i = 1}^{n} {QS}_{i} ) + α \times \sqrt{\frac{{\sum }_{i = 1}^{n} {( {QS}_{i} - mean ( {\sum }_{i = 1}^{n} QS ) )}^{2}}{n}} \\ 0 ( normal ) , otherwise & \end{matrix}$$

QSᵢ is the quantitative signal RCᵢ or DPᵢ at bin i;
QS′ᵢ is the new quantitative signal obtained from QSᵢ;
α ranging between 0.5-2 indicates the significance of the relative difference between QSᵢ and mean QS of all bins across the CDW, b=0.5 if α ranging between 0.5 and 1.5, b=1 if α ranging between 0.5 and 2; and
n is the total number of bins of interest in the CDW;
(D) perc_{chr}_{ₖ} is defined as:

$${perc}_{c h {r}_{k}} = \frac{R P {M}_{c h {r}_{k}}}{Total RPM} \times 100 with {RPM}_{c h {r}_{k}} = \frac{\frac{read {counts}_{c h {r}_{k}}}{{I}_{c h {r}_{k}}}}{{\sum }_{j = 1}^{23} \frac{read {counts}_{c h {r}_{k}}}{{I}_{c h {r}_{k}}}}$$

l is the length of chromosome of interest;
RPM_{chr}_{ₖ} is the normalized read counts of chromosome of interest in the sample of interest; and
Total RPM is the total normalized number of read counts in the sample of interest.

At step 140c, fine-tuning models for detecting fetal CNV by performing the steps (i)-(vii) for determining a set of parameters for a specific CNV;
(i) one by one through each defined CDW for each CNV at step 140a, for which one can apply a strategy of sliding window of at least 1 Mb with seeding length of four consecutive bins;
(ii) calculating the relative differences of RCᵢ and DPᵢ at each bin i in the CDW, combined with features from RCᵢ, DPᵢ and perc_{chr}_{ₖ}, which is calculated independently from said chosen CDW;
(iii) choosing levels of significant relative difference for RCᵢ and DPᵢ by comparing RCᵢ and/or DPᵢ at each bin at step (ii) to the mean of all RCᵢ and/or DPᵢ in the CDW;
(iv) one by one choosing any set of features from the plurality of quantitative signals including RCᵢ, DPᵢ, relative differences of RCᵢ and/or DPᵢ compared to mean RCᵢ and/or DPᵢ across all bins of the CDW, and perc_{chr}_{ₖ} for the learning model;
(v) one by one applying any learning model on the training set, with three parameters including the size of CDW at step (i), the level of significance aor binaries encoding for RCᵢ and/or DPᵢ determined at step (iii), and the set of features chosen from quantitative signals at step (iv), and with the hyperparameters of the learning model defined during the training process;
wherein the learning model is selected from the group consisting of machine learning-based models, Gaussian Mixture Model (GMM), and Hidden Markov Model;
wherein machine learning-based models include Naïve Bayes, K-Nearest Neighbors, Random Forest, Multi-Layer Perceptron, Support Vector Machines, and any other known machine learning-based models;
(vi) applying the trained model at step (v) to predict the CNV on the test set and evaluating if the model achieves the targeted performance metrics;
   wherein the targeted performance metrics include:
      area under the curve (AUC) is from 0.9 and above;
      accuracy is from 0.9 and above;
      sensitivity is from 0.9 and above;
      specificity is from 0.95 and above;
      positive predictive value is from 0.75 and above; and
      mean squared error (MSE) is below 0.2;
(vii) Rank the models that satisfy the targeted performance metrics and choose the best performance model as the final model for the CNV of interest; if no model is found to satisfy the targeted performance metrics, steps (iii) to (vii), or steps (iv) to (vii), or steps (v) to (vii), or steps (i) to (vii) will be repeated until a model is found.

Finally, at step 150, applying the final model to predict the CNVs on new samples, including microdeletion syndromes, or microduplications, or aneuploidies, or the number of sex chromosomes, using at least three parameters including the size of CDW, the level of significance α or binaries encoding for RCᵢ and/or DPᵢ, and the set of features chosen from quantitative signals;
wherein chromosomal aneuploidies including, but not limited to, Down syndrome (trisomy 21 or T21), Edward syndrome (trisomy 18 or T18), and Patau syndrome (trisomy 13 or T13);
wherein the microdeletion syndromes including, but not limited to, DiGeorge syndrome, Wolf-Hirschhorn syndrome, Cri-du-chat syndrome, and Prader-Willis/Angelman syndrome;
wherein the sex chromosomes include chromosome X and chromosome Y.

According to a preferred embodiment of the present invention, the final model for CNV detection is of machine learning-based models.

In a particularly advantageous embodiment of the present invention, the machine learning-based models for the number of sex chromosome detection are Multi-Layer Perceptron regression models.

In a particularly advantageous embodiment of the present invention, the machine learning-based models for detecting genetic abnormality are Support Vector Machines models.

According to embodiment of the present invention, the CDW is selected from the regions consisting of one or more of chromosome 22q11.2 region (corresponding to DiGeorge syndrome), chromosome 15q11-q13 region between positions 22-28 Mb (corresponding to Prader Willi/Angelman syndrome), chromosome 4q12 region and/or chromosome 4p16.3 region (corresponding to Wolf-Hirschhorn syndrome), and chromosome 5p15.2-p15.33 region (corresponding to Cri-du-chat syndrome), whole chromosome 13 (corresponding to T13), whole chromosome 18 (corresponding to T18), whole chromosome 21 (corresponding to T21), whole chromosome X (for estimating the number of X), and whole chromosome Y (for estimating the number of Y).

In the present invention, the chromosome 22q11.2 region comprises of one or more loci that represent the markers located in each of the inter-LCR22A-B region, the inter-LCR22A-C region, the inter-LCR22A-D region, the inter-LCR22B-D region, and the inter-LCR22C-D region;
the inter-LCR22A-B region is located between positions 19,000,000-20,500,000 bp on chromosome 22, including at least one marker from the list consisting of: PRODH, SLC25A1, CDC45, GP1BB, TBX1, TANGO2, and a combination thereof;
the inter-LCR22A-C region is located between positions 19,000,000-21,000,000 bp on chromosome 22, including at least one marker from the list consisting of: PRODH, SLC25A1, CDC45, GP1BB, TBX1, TANGO2, SCARF2, and a combination thereof;
the inter-LCR22A-D region is located between positions 19,000,000-21,500,500 bp on chromosome 22, including at least one marker from the list consisting of: PRODH, SLC25A1, CDC45, GP1BB, TBX1, TANGO2, SCARF2, SNAP29, LZTR1, and a combination thereof;
the inter-LCR22B-D region is located between positions 20,500,000-21,500,000 bp on chromosome 22, including at least one marker from the list consisting of: SCARF2, SNAP29, LZTR1, and a combination thereof;
the inter-LCR22C-D region is located between positions 21,000,000-21,500,000 bp on chromosome 22, including at least one marker from the list consisting of SNAP29, LZTR1, and a combination thereof.

In the present invention, the 5p15.2-p15.33 region between positions 0.15-11.41 Mb on chromosome 5, including at least one marker selected from the list consisting of: CEP72, TPPP, TERT, SLC6A3, MRPL36, NDUFS6, MED10, ADCY2, MTRR, SEMASA, CCT5, CTNND2, and a combination thereof.

In a particularly advantageous embodiment of the present invention, the final model for DiGeorge syndrome is a Support Vector Machines model trained with the set of features comprising the relative count (RCᵢ), and the differences between RCᵢ and mean RC of all bins in the CDW encoded in binaries (0 and 1) or encoded in levels of significant difference α.

According to a preferred embodiment of the present invention, the final model for DiGeorge syndrome is a Support Vector Machines model trained with the set of features chosen from the differences between RCᵢ and mean RC of all bins in the CDW encoded in the level of significant difference α.

In a particularly advantageous embodiment of the present invention, the final model for aneuploidies is a Support Vector Machines model trained with the set of features including the relative counts (RCᵢ), the differences between RCᵢ and mean RC of all bins in the CDW encoded in levels of significant difference α, the percentage of read counts of chromosomes of interest in the sample of interest over the total number of read counts in the sample of interest per_{chr}_{ₖ}, and the difference in proportion (DPᵢ).

According to a preferred embodiment of the present invention, the final model for aneuploidies is a Support Vector Machines model with the set of features including the percentage of read counts of chromosomes of interest in the sample of interest over the total number of read counts in the sample of interest perc_{chr}_{ₖ}, and the difference in proportion (DPᵢ).

In a particularly advantageous embodiment of the present invention, the final model for number of sex chromosome detection is a Multi-Layer Perceptron model trained with the set of features including the relative counts (RCᵢ), the differences between RCᵢ and mean RC of all bins in the CDW encoded in levels of significant difference α, the percentage of read counts of chromosomes of interest in the sample of interest over the total number of read counts in the sample of interest perc_{chr}_{ₖ}, and the difference in proportion (DPᵢ).

According to a preferred embodiment of the present invention, the final model for number of sex chromosome detection is a Multi-Layer Perceptron model trained with the set of features including the percentage of read counts of chromosomes of interest in the sample of interest over the total number of read counts in the sample of interest perc_{chr}_{ₖ}, and the difference in proportion (DPᵢ).

According to embodiment of the present invention, the method 100 further comprises training data for machine learning models generated by subsampling reads to simulate new biological samples or by data point simulation using the distribution estimated by the Gaussian Mixture Model (GMM), preferably said training data for machine learning models for microdeletion syndromes, preferably DiGeorge syndromes.

According to embodiment of the present invention, training data for machine learning models generated by subsampling reads to simulate new biological samples; wherein subsampling reads from real biological samples must ensure a limited percentage of overlapping reads between any two simulated samples is 10%, or 20%, or 30%, or 40%, or 50%, or 60%, or 70%, or 80%, or 90% of the total number of reads in each newly simulated sample; and
wherein a number of positive and negative new data points needed for machine learning-based models is selected one from the groups including from 50, from 100, from 150, from 200, from 250, from 300.

According to another embodiment of the present invention, training data for machine learning models generated by data point simulation using the distribution estimated by the Gaussian Mixture Model (GMM);
wherein a new data point simulation by GMM-based distribution estimation is done from 10, from 20, from 30, from 40, and from 50 original data points; and
wherein a number of positive and negative new data points needed for machine learning-based models is selected one from the groups including from 50, from 100, from 150, from 200, from 250, from 300.

According to embodiment of the present invention, the method 100 further comprises principal component analysis (PCA) correction to remove unwanted variations between fetal CNV-free samples, preferably said principal component analysis (PCA) correction to remove unwanted variations between fetal CNV-free samples for aneuploidies.

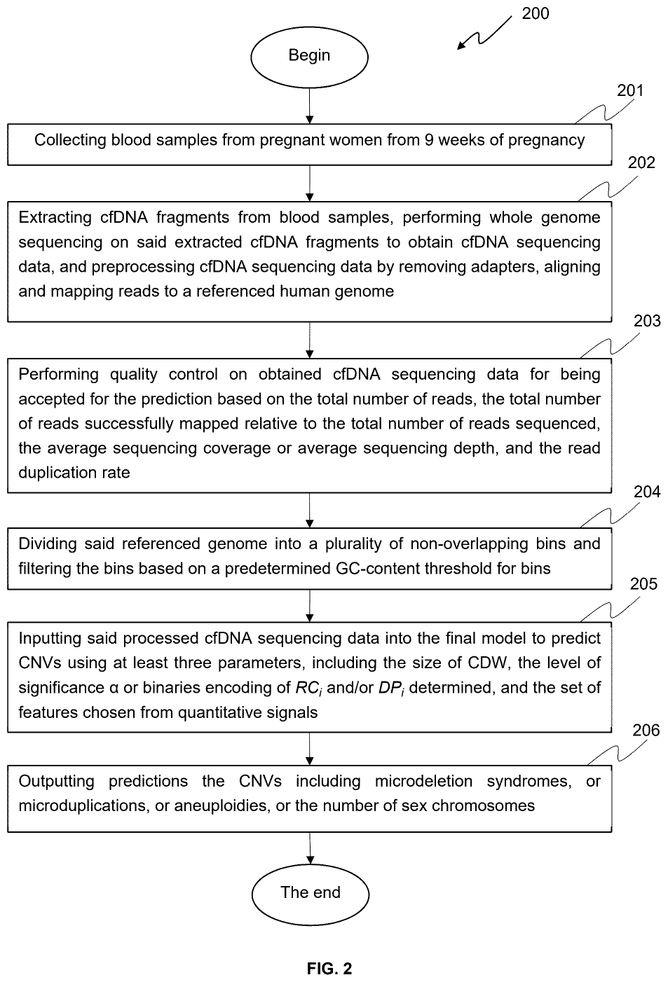

Referring to FIG. 2, the method for detecting fetal CNV through NIPT by applying the final model to predict the CNVs on new sample including microdeletion syndromes, or microduplications, or aneuploidies, or the number of sex chromosomes 200 (“method 200”) in accordance with an exemplary embodiment of the present invention, in which the final model is selected by method 100 has been described in detail above. In particular, method 200 includes the following steps:

At step 201, collecting blood samples from pregnant women from 9 weeks of pregnancy.

At step 202, extracting cell-free deoxyribonucleic acid fragments (cfDNA fragments) from blood samples, performing whole genome sequencing (WGS) on said extracted cfDNA fragments to obtain cfDNA sequencing data, and preprocessing cfDNA sequencing data by removing adapters, aligning and mapping reads to a referenced human genome.

At step 203, performing quality control on obtained cfDNA sequencing data for being accepted for the prediction based on the total number of reads, the total number of reads successfully mapped relative to the total number of reads sequenced, the average sequencing coverage or average sequencing depth, and the read duplication rate;
in which the total number of reads is of at least 18M paired-end reads;
the total number of reads successfully mapped relative to the total number of reads sequenced is at least 70%;
the average sequencing coverage or average sequencing depth is between 0.1× and 5×; and
the read duplication rate is below 30%.

According to the preferred embodiment of the present invention, the average sequencing coverage (average sequencing) depth can vary from 0.1× to 1.2×.

According to embodiment of the present invention, the step 203 further comprises samples with the obtained cfDNA sequencing data violates one or several quality control parameters, in which the total number of reads is of at least 18M paired-end reads, the average sequencing coverage or average sequencing depth is depth below 0.1×, the total number of reads successfully mapped relative to the total number of reads sequenced is less than 70%, and the read duplication rate is more than 30%. In the present invention, the sample having outlier QC metrics are rerun for sequencing, or blood samples are noted for recollection.

At step 204, dividing a referenced genome into a plurality of non-overlapping bins and filtering the bins based on a predetermined GC-content threshold for bins, wherein the bins on said referenced genome have size ranging from 50-5000 Kb.

According to the embodiment of the invention, the predetermined GC-content thresholds for bins are 30% and 70%, meaning that bins having GC-content between 30% and 70% are kept, and bins having GC-content below 30% or over 70% are removed.

At step 205, inputting said processed cfDNA sequencing data at step 204 into the final model to predict based on three parameters including the size of CDW, the level of significance α or binaries encoding for RCᵢ and/or DPᵢ determined, and the set of features chosen from quantitative signals.

In a particularly advantageous embodiment of the present invention, step 205, the final model for the number of sex chromosome detection is Multi-Layer Perceptron regression models.

In a particularly advantageous embodiment of the present invention, the final model for detecting fetal genetic abnormality is Support Vector Machines model.

In a particularly advantageous embodiment of the present invention, the final model for DiGeorge syndrome is a Support Vector Machines model trained with the set of features comprise the relative count (RCᵢ), and the differences between RCᵢ and mean RC of all bins in the CDW encoded in binaries (0 and 1) or encoded in levels of significant difference α.

According to a preferred embodiment of the present invention, the final model for DiGeorge syndrome is a Support Vector Machines model trained with the set of features chosen from the differences between RCᵢ and mean RC of all bins in the CDW encoded in the level of significant difference α.

In a particularly advantageous embodiment of the present invention, the final model for aneuploidies are Support Vector Machines models trained with the set of features including the relative counts (RCᵢ), the differences between RCᵢ and mean RC of all bins in the CDW encoded in levels of significant difference α, the percentage of read counts of chromosomes of interest in the sample of interest over the total number of read counts in the sample of interest (per_{chr}_{ₖ}), and the difference in proportion (DPᵢ).

According to a preferred embodiment of the present invention, the final model for aneuploidies are Support Vector Machines models trained with the set of features including the percentage of read counts of chromosomes of interest in the sample of interest over the total number of read counts in the sample of interest perc_{chr}_{ₖ}, and the difference in proportion (DPᵢ).

In a particularly advantageous embodiment of the present invention, the final model for number of sex chromosomes detection are Multi-Layer Perceptron regression models trained with the set of features including the relative counts (RCᵢ), the differences between RCᵢ and mean RC of all bins in the CDW encoded in levels of significant difference α, the percentage of read counts of chromosomes of interest in the sample of interest over the total number of read counts in the sample of interest perc_{chr}_{ₖ}, and the difference in proportion (DPᵢ).

According to a preferred embodiment of the present invention, the final model for number of sex chromosomes detection are Multi-Layer Perceptron regression models trained with the set of features including the percentage of read counts of chromosomes of interest in the sample of interest over the total number of read counts in the sample of interest perc_{chr}_{ₖ}, and the difference in proportion (DPᵢ).

Finally, at step 207, outputting prediction results of the CNVs including microdeletion syndromes, or microduplications, or aneuploidies, or the number of sex chromosomes.

Reference is now made to the following examples, which together with the above descriptions, illustrate the invention in a non limiting fashion.

Example 1: Selecting the final model for DiGeorge syndrome, which is a Support Vector Machines achieving the targeted performance metrics according to method 100.

Collecting N blood samples of pregnant women from at 9 weeks of pregnancy and numbering them 1-N, wherein each sample is treated corresponding to the steps of method 100, characterized by:
(A1) choosing the CDW is the chromosome 22 region;
(B1) filtering bins of a referenced genome not overlapping with the DiGeorge CDW, retaining the following set of 661 bins: chr22_1, chr22_2, chr22_3, chr22_4, . . . , chr22_660, and chr22_661.

Table 1. The results of bin filtering by overlapping bin coordination with DiGeorge CDW according to embodiment of the present invention

| Chromosome | Start | End | GC | Mappability |
|---|---|---|---|---|
| 22 | 16500001 | 16550000 | 44.34 | 38.85 |
| 22 | 16550001 | 16600000 | 45.47 | 40.23 |
| 22 | 16600001 | 16650000 | 41.24 | 33.89 |
| . . . | . . . | . . . | . . . | . . . |
| 22 | 50650001 | 50700000 | 59.13 | 87.72 |
| 22 | 50700001 | 50750000 | 64.72 | 94.62 |
| 22 | 50750001 | 50800000 | 53.70 | 75.36 |

(C1) Calculating read counts at each bin of said kept bins and simultaneously calculates similarly on the corresponding N samples, the results of step (C1) are listed by Table 2 below.

Table 2. The results of read counts at kept bins on per sample from 1-N according to embodiment of the present invention

| Sample | Bin 1 | Bin 2 | . . . | Bin 661 |
|---|---|---|---|---|
| Sample 1 | 1 | 2 | . . . | 4 |
| Sample 2 | 2 | 3 | . . . | 1 |
| . . . | . . . | . . . | . . . | . . . |
| Sample N | 5 | 10 | . . . | 2 |

(D1) Calculating fetal relative counts at each bin of said kept bins and simultaneously calculates similarly on the corresponding N samples, the results of step (D1) are listed by Table 3 below.

Table 3. The results of fetal relative counts of each kept bin on per sample from 1-N according to embodiment of the present invention

| Samples | Bin 1 | Bin 2 | . . . | Bin 661 |
|---|---|---|---|---|
| Sample 1 | 0.00001 | 0.00002 | . . . | 0.00004 |
| Sample 2 | 0.00002 | 0.00003 | . . . | 0.00001 |
| . . . | . . . | . . . | . . . | . . . |
| Sample N | 0.000001 | 0.000001 | . . . | 0.0000002 |

(E1) transforming the read counts of said kept bins and simultaneously calculates similarly on the corresponding N samples into quantitative signals encoded in the level of significant difference α, with α∈{O, 1, 2}, and b=1, the results of step (E1) are listed by Table 4 below.

Table 4. The results of transforming the read counts of each kept bin on per sample from 1-N into quantitative signals encoded in the level of significant difference α according to embodiment of the present invention.

| Sample | Bin 1 | Bin 2 | . . . | Bin 661 |
|---|---|---|---|---|
| Sample 1 | 1 | 2 | . . . | 1 |
| Sample 2 | 3 | 1 | . . . | 2 |
| . . . | . . . | . . . | . . . | . . . |
| Sample N | 2 | 1 | . . . | 3 |

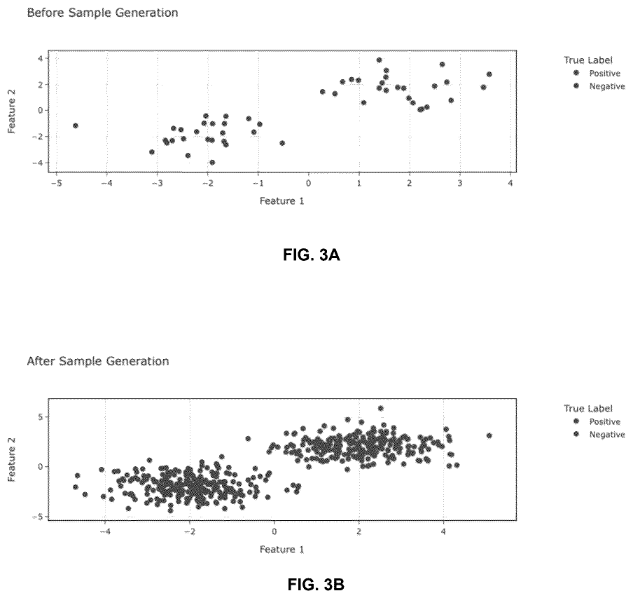

(F1) generating training data for Support Vector Machines by data point generation using the distribution estimated by Gaussian Mixture Model (GMM). The training data generation will be described by way of example according to FIG. 3a-FIG. 3b illustrating the training dataset in reduced feature space of N sample before and after data point generation using the distribution estimated by GMM, in which the number of said feature space is two.

(G1) inputting the training data into the Support Vector Machines (SVM) model for training to classify the normal samples (negative, Dieorge-free) and abnormal samples (positive, Dieorge-detected), in which said training process is repeated many times until the SVM model obtain the targeted performance metrics, referring pre-trained SVM model's performance on the test set listed in Table 5 below.

Table 5. Performance of the SVM model for DiGeorge screening on the test set

| Metrics | Test set/Clinically validated data |
|---|---|
| Sensitivity | 85.71% |
| Specificity | 100% |
| Accuracy | 99.64% |
| AUC | 99.89% |

(H1) Prediction results of the possibility of fetal DiGeorge on new samples through Support Vector Machines model listed in Table 6 below.

Table 6. Prediction results of the possibility of fetal DiGeorge on new samples through pre-trained Support Vector Machines model

| Lab- | Positive | Label | | Positive | Label |
|---|---|---|---|---|---|
| code | probability | prediction | Labcode | probability | prediction |
| S405 | 0.029887128 | 0 | S394 | 0.00782325 | 0 |
| S401 | 0.04417077 | 0 | S393 | 0.132085399 | 0 |
| S397 | 0.01069398 | 0 | S402 | 0.006338739 | 0 |
| S379 | 0.003594639 | 0 | S380 | 0.012691752 | 0 |
| S403 | 0.007944728 | 0 | S381 | 0.006427279 | 0 |
| S392 | 0.011363961 | 0 | S385 | 0.016509253 | 0 |
| S395 | 0.012823201 | 0 | S399 | 0.01945896 | 0 |
| S384 | 0.9999871 | 1 | S406 | 0.009138346 | 0 |
| S404 | 0.059667316 | 0 | S400 | 0.005777262 | 0 |
| S388 | 0.029351923 | 0 | S398 | 0.002739842 | 0 |
| S396 | 0.004480529 | 0 | S391 | 0.010427145 | 0 |
| S383 | 0.004569715 | 0 | S387 | 0.004573856 | 0 |
| S382 | 0.003807525 | 0 | S386 | 0.006457259 | 0 |

Example 2: Selecting the final model for Trisomy 21 (T21), which is a Support Vector Machines model achieving the targeted performance metrics according to method 100.

Collecting N blood samples of pregnant women from at 9 weeks of pregnancy and numbering them 1-N, wherein each sample is treated corresponding to the steps of method 100, characterized by:
(A2) choosing the CDW is chromosome 21 region;
(B2) filtering bins of a referenced genome not overlapping with the T21 CDW listed in Table 7 below.

Table 7. The results of bin filtering by overlapping bin coordination with T21 CDW according to embodiment of the present invention

| Chromosome | Start | End | GC | Mappability |
|---|---|---|---|---|
| 21 | 900001 | 950000 | 36.81 | 31.66 |
| 21 | 10500001 | 1100000 | 38.93 | 46.43 |
| 21 | 1100001 | 1150000 | 40.46 | 55.41 |
| . . . | . . . | . . . | . . . | . . . |
| 21 | 45000001 | 45500000 | 50.10 | 87.24 |
| 21 | 45500001 | 46000000 | 54.77 | 89.16 |
| 21 | 46000001 | 46500000 | 52.21 | 90.55 |

NaN: Undetermined

(C2) Calculating fetal read counts at each bin of said kept bins and simultaneously calculates similarly on the corresponding N samples, the results of step (C2) are listed by Table 8 below.

Table 8. The results of fetal read counts of each kept bin on per sample from 1-N according to embodiment of the present invention

| Sample | Bin 1 | Bin 2 | . . . | Bin 67 |
|---|---|---|---|---|
| Sample 1 | 1 | 2 | . . . | 4 |
| Sample 2 | 2 | 3 | . . . | 1 |
| . . . | . . . | . . . | . . . | . . . |
| Sample N | 5 | 10 | . . . | 2 |

(D2) Calculating difference in proportion (DPᵢ) at each bin of said kept bins and simultaneously calculates similarly on the corresponding N samples, the results of step (D2) are listed by Table 9 below.

Table 9. The results of difference in proportion (DPᵢ) at each bin of said kept bins on per sample from 1-N according to embodiment of the present invention

| Sample | Bin 1 | Bin 2 | . . . | Bin 67 |
|---|---|---|---|---|
| Sample 1 | 0.0077 | 0.0061 | . . . | 0.002 |
| Sample 2 | 0.0008 | −0.004 | . . . | 0.004 |
| . . . | . . . | . . . | . . . | . . . |
| Sample N | 0.024 | 0.0249 | . . . | 0.022 |

(E2) calculating all chromosome read counts, chromosome read counts normalized by RPM, and per_{chr}_{ₖ} at each chromosome from 24 chromosomes (X and Y chromosomes are counted independently) on the corresponding N samples, all listed by Table 10-12 below.

Table 10. The results of all chromosome read counts on the corresponding N samples according to embodiment of the present invention

| Sample | Chromosome 1 | Chromosome 2 | . . . | Chromosome Y |
|---|---|---|---|---|
| Sample 1 | 195740 | 114552 | . . . | 2726 |
| Sample 2 | 231503 | 141112 | . . . | 3372 |
| . . . | . . . | . . . | . . . | . . . |
| Sample N | 173556 | 104213 | . . . | 3326 |

Table 11. The results of all chromosome read counts normalized by RPM on the corresponding N samples according to embodiment of the present invention

| Sample | Chromosome 1 | Chromosome 2 | . . . | Chromosome Y |
|---|---|---|---|---|
| Sample 1 | 45026.92 | 49031.03 | . . . | 2727.95 |
| Sample 2 | 44299.05 | 50243.21 | . . . | 2807.01 |
| . . . | . . . | . . . | . . . | . . . |
| Sample N | 44449.35 | 50122.47 | . . . | 3282.39 |

Table 12. The results of the per_{chr}_{ₖ} on the corresponding N samples according to embodiment of the present invention

| Sample | Chromosome 1 | Chromosome 2 | . . . | Chromosome Y |
|---|---|---|---|---|
| Sample 1 | 4.56 | 4.96 | . . . | 0.28 |
| Sample 2 | 4.51 | 5.03 | . . . | 0.29 |
| . . . | . . . | . . . | . . . | . . . |
| Sample N | 4.58 | 5.16 | . . . | 0.33 |

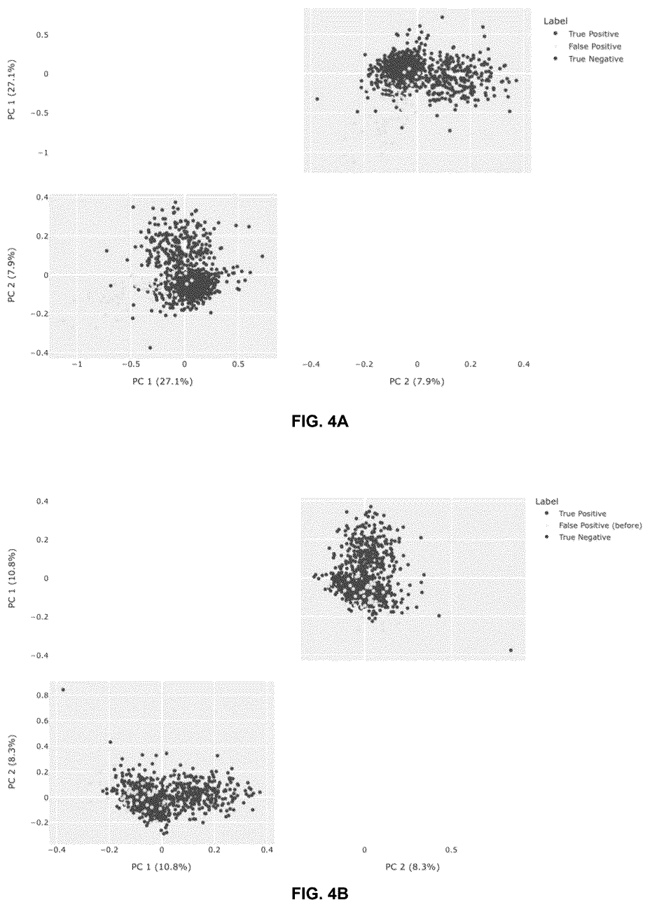

(F2) removing unwanted variations between fetal CNV-free samples by performing principal component analysis (PCA) correction on training data. The PCA correction on training data will be described by way of example according to FIG. 4a-FIG. 4b illustrating the training dataset in reduced feature space of N sample. In FIG. 4a, CNV-free data points (yellow) containing unwanted variation are located far from other CNV-free data points (green). In FIG. 4b, CNV-free samples (yellow) containing unwanted variation are located among other CNV-free samples (green) thanks to PCA correction. For industrial application, the corrected loadings for PCA will be re-used to remove unwanted variation on new predicted samples.

(G2) inputting the corrected training data into the Support Vector Machines model for training to classify the T21-free samples (negative) or T21-detected samples (positive), in which said training process is repeated many times until the SVM model obtain the targeted performance metrics, referring pre-trained SVM model's results on the test set listed in Table 13 below.

Table 13. Performance of the SVM model for T21 screening on the test set

| Metrics | Test set |
|---|---|
| Sensitivity | 96.78% |
| Specificity | 96.58% |
| Accuracy | 96.72% |
| AUC | 96.68% |

(H2) Prediction results of the possibility of fetal T21 on new samples through Support Vector Machines model listed in Table 14 below.

Table 14. Prediction results of the possibility of fetal T21 on new samples through Support Vector Machines model

| | | T21 | | | T21 |
|---|---|---|---|---|---|
| | T21 | Positive | | T21 | Positive |
| Labcode | Prediction | Probability | Labcode | Prediction | Probability |
| S385 | 0 | 0.001 | S401 | 0 | 0.002 |
| S382 | 0 | 0.023 | S392 | 0 | 0.005 |
| S380 | 0 | 0.002 | S400 | 0 | 0.004 |
| S397 | 0 | 0.005 | S396 | 0 | 0.011 |
| S384 | 0 | 0.001 | S398 | 0 | 0.004 |
| S381 | 0 | 0.007 | S406 | 0 | 0.005 |
| S387 | 0 | 0.013 | S386 | 0 | 0.003 |
| S404 | 0 | 0.018 | S403 | 0 | 0.002 |
| S383 | 0 | 0.002 | S391 | 0 | 0.004 |
| S393 | 0 | 0.009 | S388 | 0 | 0.003 |
| S399 | 0 | 0.019 | S402 | 0 | 0.007 |
| S405 | 0 | 0.001 | S394 | 0 | 0.007 |
| S395 | 0 | 0.004 | S379 | 0 | 0.006 |
| S64 | 1 | 0.998 | | | |

Example 3: Selecting the final model for number of sex chromosome is a Multi-Layer Perceptron achieves the targeted performance metrics according to method 100.

Collecting N blood samples of pregnant women from at 9 weeks of pregnancy and numbering them 1-N, wherein each sample is treated corresponding to the steps of method 100, characterized by:
(A3) choosing the CDW is chromosome X region;
(B3) filtering bins of a referenced genome not overlapping with the chromosome X CDW listed in Table 15 below.

Table 15. The results of bin filtering by overlapping bin coordination with chromosome X CDW according to embodiment of the present invention

| Chromosome | Start | End | GC | Mappability |
|---|---|---|---|---|
| X | 1 | 500000 | 52.99 | 31.37 |
| X | 500001 | 1000000 | 45.83 | 37.28 |
| X | 1000001 | 1500000 | 48.17 | 35.96 |
| . . . | . . . | . . . | . . . | . . . |
| X | 154000001 | 154500001 | 39.61 | 82.64 |
| X | 154500001 | 155000001 | 38.40 | 63.63 |
| X | 155000001 | 155270560 | 41.41 | 37.83 |

NaN: Undetermined

(C3) Calculating fetal read counts at each bin of said kept bins and simultaneously calculates similarly on the corresponding N samples listed by Table 16 below.

Table 16. The results of fetal read counts of each kept bin on per sample from 1-N according to embodiment of the present invention

| Sample | Bin 1 | Bin 2 | . . . | Bin 305 |
|---|---|---|---|---|
| Sample 1 | 1 | 2 | . . . | 4 |
| Sample 2 | 2 | 3 | . . . | 1 |
| . . . | . . . | . . . | . . . | . . . |
| Sample N | 5 | 10 | . . . | 2 |

(D3) Calculating difference in proportion (DPᵢ) at each bin of said kept bins and simultaneously calculates similarly on the corresponding N samples, listed by Table 17 below.

Table 17. The results of difference in proportion (DPᵢ) at each bin of said kept bins on per sample from 1-N according to embodiment of the present invention

| Sample | Bin 1 | Bin 2 | . . . | Bin 305 |
|---|---|---|---|---|
| Sample 1 | 0.0077 | 0.0061 | . . . | 0.002 |
| Sample 2 | 0.0008 | −0.004 | . . . | 0.004 |
| . . . | . . . | . . . | . . . | . . . |
| Sample N | 0.024 | 0.0249 | . . . | 0.022 |

(E3) calculating all chromosome read count, all chromosome read count normalized by RPM, and per_{chr}_{ₖ} at each chromosome from 24 chromosomes (X and Y chromosomes are counted independently) on the corresponding N samples, all listed by Table 18-20 below.

Table 18. The results of all chromosome read count on the corresponding N samples according to embodiment of the present invention

| Sample | Chromosome 1 | Chromosome 2 | . . . | Chromosome Y |
|---|---|---|---|---|
| Sample 1 | 195740 | 114552 | . . . | 2726 |
| Sample 2 | 231503 | 141112 | . . . | 3372 |
| . . . | . . . | . . . | . . . | . . . |
| Sample N | 173556 | 104213 | . . . | 3326 |

Table 19. The results of all chromosome read count normalized by RPM on the corresponding N samples according to embodiment of the present invention

| Sample | Chromosome 1 | Chromosome 2 | . . . | Chromosome Y |
|---|---|---|---|---|
| Sample 1 | 45026.92 | 49031.03 | . . . | 2727.95 |
| Sample 2 | 44299.05 | 50243.21 | . . . | 2807.01 |
| . . . | . . . | . . . | . . . | . . . |
| Sample N | 44449.35 | 50122.47 | . . . | 3282.39 |

Table 20. The results of the per_{chr}_{ₖ} on the corresponding N samples according to embodiment of the present invention

| Sample | Chromosome 1 | Chromosome 2 | . . . | Chromosome Y |
|---|---|---|---|---|
| Sample 1 | 4.56 | 4.96 | . . . | 0.28 |
| Sample 2 | 4.51 | 5.03 | . . . | 0.29 |
| . . . | . . . | . . . | . . . | . . . |
| Sample N | 4.58 | 5.16 | . . . | 0.33 |

(F3) inputting the training data into the Multi-Layer Perceptron regression model for training for the X chromosome detection, in which said training process is repeated many times until the Multi-Layer Perceptron model achieves the targeted performance metrics, referring pre-trained Multi-Layer Perceptron model's result on the test set listed in Table 21 below.

Table 21. Performance of the Multi-Layer Perceptron regression model for the X chromosome detection on the test set

| Metrics | Test set |
|---|---|
| Mean Square Error | 0.0022 |

(G3) Prediction results of the number of X (or Y) chromosome on new samples through Multi-Layer Perceptron model listed in Table 22 below.

Table 22. Prediction results of the X (or Y) chromosome detection on new samples through Multi-Layer Perceptron model

Inferred Number of Y

| | | | | (should be |
|---|---|---|---|---|
| | | | Inferred | interpreted |
| | | | Number of | Inferred |
| | | | X (should be | Number |
| | X | Y | interpreted | of Y by |
| Labcode | Regression | Regression | by physician) | physician) |
| S393 | 1.023057993 | 0.945436566 | 1 | 1 |
| S391 | 1.013345421 | 0.504974459 | 1 | 1 |
| S401 | 2.019794193 | −0.073300874 | 2 | 0 |
| S404 | 1.021947585 | 0.886959423 | 1 | 1 |
| S400 | 1.985167901 | −0.095752966 | 2 | 0 |
| S392 | 1.679189604 | −0.083963914 | 2 | 0 |
| S382 | 1.998119855 | −0.075303682 | 2 | 0 |
| S387 | 2.007226914 | −0.077232944 | 2 | 0 |
| S405 | 2.036242531 | −0.090379327 | 2 | 0 |
| S381 | 1.015854351 | 0.918149029 | 1 | 1 |
| S395 | 1.869340429 | −0.077137748 | 2 | 0 |
| S379 | 2.028463849 | −0.065512176 | 2 | 0 |
| S402 | 1.027998334 | 1.054572302 | 1 | 1 |
| S293 | 1.041939 | 0.101969 | 1 | 0 |
| S383 | 1.993763079 | −0.078784799 | 2 | 0 |
| S399 | 1.027210167 | 0.43358726 | 1 | 0 |
| S406 | 1.999672922 | −0.076360443 | 2 | 0 |
| S385 | 1.950045615 | −0.095633956 | 2 | 0 |
| S398 | 1.004774485 | 0.657169303 | 1 | 1 |
| S384 | 2.012096169 | −0.085303899 | 2 | 0 |
| S403 | 2.052503734 | −0.078182471 | 2 | 0 |
| S397 | 1.011940159 | 0.885261084 | 1 | 1 |
| S386 | 1.954302963 | −0.094305169 | 2 | 0 |
| S396 | 1.985396972 | −0.097385675 | 2 | 0 |
| S388 | 1.012938714 | 0.742862213 | 1 | 1 |
| S380 | 1.089816802 | 0.578251449 | 1 | 1 |
| S394 | 2.049860643 | −0.073596444 | 2 | 0 |

According to another embodiment of the invention, a system for evaluation of fetal CNV in a test sample through NIPT, the system comprising: a sequencer for receiving cell-free deoxyribonucleic acid fragments (cfDNA fragments) from the test sample and providing cfDNA sequencing data of the test sample;
a computer; and
one or more computer-readable storage media having stored thereon instructions for execution on said computer to:
(a) collecting blood samples from pregnant women from 9 weeks of pregnancy;
(b) extracting cell-free deoxyribonucleic acid fragments (cfDNA fragments) from blood samples, performing whole genome sequencing (WGS) on said extracted cfDNA fragments to obtain cfDNA sequencing data, and preprocessing cfDNA sequencing data by removing adapters, aligning and mapping reads to a referenced human genome;
(c) performing quality control on obtained cfDNA sequencing data for being accepted for the prediction based on the total number of reads, the total number of reads successfully mapped relative to the total number of reads sequenced, the average sequencing coverage or average sequencing depth, and the read duplication rate;
   in which the total number of reads is of at least 18M paired-end reads;
   the total number of reads successfully mapped relative to the total number of reads sequenced is at least 70%;
   the average sequencing coverage or average sequencing depth is between 0.1× and 5×; and the read duplication rate is below 30%;
(d) dividing a referenced genome into a plurality of non-overlapping bins and filtering the bins based on a predetermined GC-content threshold for bins;
   wherein the bins on said referenced genome have size ranging from 50-5000 Kb;
   wherein bins having GC-content between 30 and 70 are kept, and bins having GC-content below 30 or over 70 are removed;
(e) defining a CNV detection window (CDW), a bin size, a set of features for machine learning/deep learning models and fine-tune said model for detecting fetal copy number variation for selecting a final model, comprising the steps of:
   (Step 1) Defining a plurality of CDWs, wherein a CDW is defined for each CNV type or subtype of interest, wherein a CDW encompasses a region on the genome where the CNV of interest is located, wherein a CDW is the region of CNV of interest, or of at least 50% bigger than the region of CNV of interest, in which bins are discarded unless located within the defined CDW;
   (Step 2) Preparing a set of features for model learning by transforming the raw read counts of kept bins at (step 1) into a set of quantitative copy number signals at bins of interest, then using these quantitative signals as features for a model, allowing the model to learn from the features to detect CNV;
      in which the features is selected from the set of quantitative signals (QS), including (A) relative counts (RCᵢ with i among the plurality of bins), (B) difference in proportion (DPᵢ with i among the plurality of bins), (C) relative differences of RCᵢ or DPᵢ and the mean RC or DP across all bins in the window of detection, and (D) percentages of read counts of chromosomes of interest in the sample of interest over the total number of read counts in the sample of interest perc_{ch}_{ₖ};
      (A) RCᵢ is defined as:

$$R {C}_{i} = \frac{fetal read {counts}_{i}}{total read counts of sample}$$

(B) DPᵢ is defined as:

$$D {P}_{i} = \frac{fetal read {counts}_{i}}{total fetal read counts of sample} - \frac{maternal read {counts}_{i}}{total maternal read counts of sample}$$

wherein the fetal read counts correspond to the fetal fragment size, in which the fetal fragment size is below 140 base-pairs, below 145 base-pairs, below 150 base-pairs, below 155 base-pairs, below 160 base-pairs, below 160 base-pairs, or below 170 base-pairs;
wherein the maternal read counts correspond to the maternal fragment size, in which the maternal fragment size is above 160 base-pairs, above 165 base-pairs, above 170 base-pairs, and above 175 base-pairs;
(C) the relative difference between the quantitative signal RCᵢ or DPᵢ and the mean RC or DP of all bins across the CDW encoded in binaries (0 and 1) or encoded in levels of significant difference α;
wherein the relative difference between the quantitative signal RCᵢ or DPᵢ and the mean RC or DP of all bins across the CDW encoded in binaries (0 and 1);

$${QS}_{i} = \{ \begin{matrix}1 ( deletion ) & if {QS}_{i} \leq mean ( {\sum }_{i = 1}^{n} {QS}_{i} ) - α \times \sqrt{\frac{{\sum }_{i = 1}^{n} {( {QS}_{i} - mean ( {\sum }_{i = 1}^{n} QS ) )}^{2}}{n}} \\ 1 ( duplication ) & if {QS}_{i} \geq mean ( {\sum }_{i = 1}^{n} {QS}_{i} ) + α \times \sqrt{\frac{{\sum }_{i = 1}^{n} {( {QS}_{i} - mean ( {\sum }_{i = 1}^{n} QS ) )}^{2}}{n}} \\ 0 ( normal ) , & otherwise\end{matrix}$$

QSᵢ is the quantitative signal RCᵢ or DPᵢ at bin i;
α ranging between 0.5-2 indicates the significance of the relative difference between QSᵢ and mean QS of all bins across the CDW; and
n is the total number of bins of interest in the CDW;
wherein the relative difference between the quantitative signal RCᵢ or DPᵢ and the mean RC or DP of all bins across the CDW encoded in levels of significant difference α:

$${QS}_{i}^{′} = \{ \begin{matrix}α + b ( deletion ) & if {QS}_{i} \leq mean ( {\sum }_{i = 1}^{n} {QS}_{i} ) - α \times \sqrt{\frac{{\sum }_{i = 1}^{n} {( {QS}_{i} - mean ( {\sum }_{i = 1}^{n} QS ) )}^{2}}{n}} \\ α + b ( duplication ) & if {QS}_{i} \geq mean ( {\sum }_{i = 1}^{n} {QS}_{i} ) + α \times \sqrt{\frac{{\sum }_{i = 1}^{n} {( {QS}_{i} - mean ( {\sum }_{i = 1}^{n} QS ) )}^{2}}{n}} \\ 0 ( normal ) , & otherwise\end{matrix}$$

QSᵢ is the quantitative signal RCᵢ or DPᵢ at bin i;
QS′ᵢ is the new quantitative signal obtained from QSᵢ;
α ranging between 0.5-2 indicates the significance of the relative difference between QSᵢ and mean QS of all bins across the CDW, b=0.5 if α ranging between 0.5 and 1.5, b=1 if α ranging between 0.5 and 2; and
n is the total number of bins of interest in the CDW;
(D) perc_{chr}_{ₖ} is defined as:

$${perc}_{{chr}_{k}} = \frac{R P {M}_{c h {r}_{k}}}{Total RPM} \times 100 with {RPM}_{{chr}_{k}} = \frac{\frac{read {counts}_{{chr}_{k}}}{{l}_{{chr}_{k}}}}{{\sum }_{j = 1}^{23} \frac{read {counts}_{{chr}_{k}}}{{l}_{{chr}_{k}}}}$$

l is the length of chromosome of interest;
RPM_{chr}_{ₖ} is the normalized read counts of chromosome of interest in the sample of interest; and
Total RPM is the total normalized number of read counts in the sample of interest.
(Step 3) fine-tuning models for detecting fetal CNV by repeating the following steps until a set of parameters is determined for a specific CNV;
   (i) one by one through each defined CDW for each CNV at (step 1), for which one can apply a strategy of sliding window of at least 1 Mb with seeding length of four consecutive bins;
   (ii) calculating the relative differences of RCᵢ and DPᵢ at each bin i in the CDW combined with features from RCᵢ, DPᵢ and perc_{chr}_{ₖ}, which is calculated independently from said chosen CDW;
   (iii) choosing levels of significant α RCᵢ and DPᵢ by comparing RCᵢ and/or DPᵢ at each bin at step (ii) to the mean of all RCᵢ and/or DPᵢ in the CDW;
   (iv) one by one choosing any set of features from the plurality of quantitative signals including RCᵢ, DPᵢ, relative differences of RCᵢ and/or DPᵢ compared to mean RCᵢ and/or DPᵢ across all bins of the CDW, and perc_{chr}_{ₖ} for the learning model;
   (v) one by one applying any learning model on the training set, with three main parameters including the size of CDW at step (i), the level of significance α or binaries encoding for RCᵢ and/or DPᵢ determined at step (iii), and the set of features chosen from quantitative signals at step (iv), and with the hyperparameters of the learning model defined during the training process;
      wherein the learning model is selected from the group consisting of machine learning-based models, Gaussian Mixture Model (GMM), and Hidden Markov Model;
      wherein machine learning-based models include Naïve Bayes, K-Nearest Neighbors, Random Forest, Multi-Layer Perceptron, Support Vector Machines, or any other known machine learning-based models;
   (vi) applying the trained model at step (v) to predict the CNV on the test set and evaluating if the model achieves the targeted performance metrics;
      wherein the targeted performance metrics include:
      area under the curve (AUC) is from 0.9 and above;
      accuracy is from 0.9 and above;
      sensitivity is from 0.9 and above;
      specificity is from 0.95 and above; and
      positive predictive value is from 0.75 and above;
      mean squared error (MSE) is below 0.2;
   (vii) Rank the models that satisfy the targeted performance metrics and choose the best performance model as the final model for the CNV of interest; if no model is found to satisfy the targeted performance metrics, steps (iii) to (vii), or steps (iv) to (vii), or steps (v) to (vii), or steps (i) to (vii) will be repeated until a model is found; and
(f) applying the final model to predict CNVs, including microdeletion syndromes, or microduplications, or aneuploidies, or the number of sex chromosomes, using at least three parameters, including the size of CDW, the level of significance α or binaries encoding for RCᵢ and/or DPᵢ, and the set of features chosen from quantitative signals;
   wherein chromosomal aneuploidies including Down syndrome (trisomy 21 or T21), Edward syndrome (trisomy 18 or T18), and Patau syndrome (trisomy 13 or T13);
   wherein the microdeletion syndromes include DiGeorge syndrome, Wolf-Hirschhorn syndrome, Cri-du-chat syndrome, and Prader-Willis/Angelman syndrome;
   wherein the sex chromosomes include chromosome X and chromosome Y.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, element components, and/or groups thereof.

While the preferred embodiment to the invention had been described, it will be understood that those skilled in the art, both now and in the future, may make various improvements and enhancements which fall within the scope of the claims which follow. These claims should be construed to maintain the proper protection for the invention first described.

The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application and to enable others of ordinary skill in the art to understand the invention for various embodiments with various modifications as are suited to the particular use contemplated.

The flow diagrams depicted herein are just one example. There may be many variations to this diagram, or the steps (or operations) described therein without departing from the spirit of the invention. For instance, the steps may be performed in a differing order, or steps may be added, deleted, or modified. All of these variations are considered a part of the claimed invention.

While the preferred embodiment to the invention had been described, it will be understood that those skilled in the art, both now and in the future, may make various improvements and enhancements which fall within the scope of the claims which follow. These claims should be construed to maintain the proper protection for the invention first described.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. A method for detecting fetal copy number variation (CNV) through non-invasive prenatal testing (NIPT), comprising steps performed in the following specific order:
(a) collecting blood samples from pregnant women from 9 weeks of pregnancy;
(b) extracting cell-free deoxyribonucleic acid fragments (cfDNA fragments) from the blood samples, performing whole genome sequencing (WGS) on said extracted cfDNA fragments to obtain cfDNA sequencing data, and preprocessing cfDNA sequencing data by removing adapters, aligning and mapping reads to a referenced human genome;
(c) performing quality control on obtained cfDNA sequencing data for being accepted for the prediction based on the total number of reads, the total number of reads successfully mapped relative to the total number of reads sequenced, the average sequencing coverage or average sequencing depth, and the read duplication rate;
in which the total number of reads is of at least 18M paired-end reads;
the total number of reads successfully mapped relative to the total number of reads sequenced is at least 70%;
the average sequencing coverage or average sequencing depth is between 0.1× and 5×; and
the read duplication rate is below 30%;
(d) dividing a referenced genome into a plurality of non-overlapping bins and filtering the bins based on a predetermined GC-content threshold for bins;
wherein the bins on said referenced genome have size ranging from 50-5000 Kb;
wherein bins having GC-content between 30 and 70 are kept, and bins having GC-content below 30 or over 70 are removed;
(e) defining a CNV detection window (CDW), a bin size, a set of features for machine learning/deep learning models and fine-tune said model for detecting fetal copy number variation for selecting a final model, comprising the steps of:
(Step 1) Defining a plurality of CDWs, wherein a CDW is defined for each CNV type or subtype of interest, wherein a CDW encompasses a region on the genome where the CNV of interest is located, wherein a CDW is the region of CNV of interest, or of at least 50% bigger than the region of CNV of interest, in which bins are discarded unless located within the defined CDW;
(Step 2) Preparing a set of features for model learning by transforming the raw read counts of kept bins at (step 1) into a set of quantitative copy number signals at bins of interest, then using these quantitative signals as features for a model, allowing the model to learn from the features to detect CNV;
in which the features is selected from the set of quantitative signals (QS), including (A) relative counts (RCᵢ with i among the plurality of bins), (B) difference in proportion (DPᵢ with i among the plurality of bins), (C) relative differences of RCᵢ or DPᵢ and the mean RC or DP across all bins in the CDW, and (D) percentages of read counts of chromosomes of interest in the sample of interest over the total number of read counts in the sample of interest perc_{chr}_{ₖ};
(A) RCᵢ is defined as:
$$R {C}_{i} = \frac{fetal read {counts}_{i}}{total read counts of sample}$$
(B) DPᵢ is defined as:
$$D {P}_{i} = \frac{fetal read {counts}_{i}}{total fetal read counts of sample} - \frac{maternal read {counts}_{i}}{total maternal read counts of sample}$$
wherein the fetal read counts correspond to the fetal fragment size, in which the fetal fragment size is below 140 base-pairs, below 145 base-pairs, below 150 base-pairs, below 155 base-pairs, below 160 base-pairs, below 160 base-pairs, or below 170 base-pairs;
wherein the maternal read counts correspond to the maternal fragment size, in which the maternal fragment size is above 160 base-pairs, above 165 base-pairs, above 170 base-pairs, and above 175 base-pairs;
(C) the relative difference between the quantitative signal RCᵢ or DPᵢ and the mean RC or DP of all bins across the CDW encoded in binaries (0 and 1) or encoded in levels of significant difference α;
wherein the relative difference between the quantitative signal RCᵢ or DPᵢ and the mean RC or DP of all bins across the CDW encoded in binaries (0 and 1);
$${QS}_{i} = \{ \begin{matrix}1 ( deletion ) if Q {S}_{i} & \leq mean ( {\sum }_{i = 1}^{n} {QS}_{i} ) - α \times \sqrt{\frac{{\sum }_{i = 1}^{n} {( {QS}_{i} - mean ( {\sum }_{i = 1}^{n} QS ) )}^{2}}{n}} \\ 1 ( duplication ) if Q {S}_{i} & \geq mean ( {\sum }_{i = 1}^{n} {QS}_{i} ) + α \times \sqrt{\frac{{\sum }_{i = 1}^{n} {( {QS}_{i} - mean ( {\sum }_{i = 1}^{n} QS ) )}^{2}}{n}} \\ 0 ( normal ) , otherwise & \end{matrix}$$
QSᵢ is the quantitative signal RCᵢ or DPᵢ at bin i;
α ranging between 0.5-2 indicates the significance of the relative difference between QSᵢ and mean QS of all bins across the CDW; and
n is the total number of bins of interest in the CDW;
wherein the relative difference between the quantitative signal RCᵢ or DPᵢ and the mean RC or DP of all bins across the CDW encoded in encoded in levels of significant difference α:
$${QS}_{i}^{′} = \{ \begin{matrix}α + b ( deletion ) if Q {S}_{i} & \leq mean ( {\sum }_{i = 1}^{n} {QS}_{i} ) - α \times \sqrt{\frac{{\sum }_{i = 1}^{n} {( {QS}_{i} - mean ( {\sum }_{i = 1}^{n} QS ) )}^{2}}{n}} \\ α + b ( duplication ) if Q {S}_{i} & \geq mean ( {\sum }_{i = 1}^{n} {QS}_{i} ) + α \times \sqrt{\frac{{\sum }_{i = 1}^{n} {( {QS}_{i} - mean ( {\sum }_{i = 1}^{n} QS ) )}^{2}}{n}} \\ 0 ( normal ) , otherwise & \end{matrix}$$
QSᵢ is the quantitative signal RCᵢ or DPᵢ at bin i;
QS′ᵢ is the new quantitative signal obtained from QSᵢ;
α ranging between 0.5-2 indicates the significance of the relative difference between QSᵢ and mean QS of all bins across the CDW, b=0.5 if α ranging between 0.5 and 1.5, b=1 if α ranging between 0 and 2; and
n is the total number of bins of interest in the CDW;
(D) perc_{chr}_{ₖ} is defined as:
$${perc}_{c h {r}_{k}} = \frac{R P {M}_{c h {r}_{k}}}{Total RPM} \times 100 with {RPM}_{c h {r}_{k}} = \frac{\frac{read {counts}_{c h {r}_{k}}}{{I}_{c h {r}_{k}}}}{{\sum }_{j = 1}^{23} \frac{read {counts}_{c h {r}_{k}}}{{I}_{c h {r}_{k}}}}$$
l is the length of chromosome of interest;
RPM_{chr}_{ₖ} is the normalized read counts of chromosome of interest in the sample of interest; and
Total RPM is the total normalized number of read counts in the sample of interest;
(Step 3) fine-tuning models for detecting fetal CNV by repeating the following steps until a set of parameters is determined for a specific CNV;
(i) one by one through each defined CDW for each CNV at (step 1), for which one can apply a strategy of sliding window of at least 1 Mb with seeding length of four consecutive bins;
(ii) calculating the relative differences of RCᵢ and DPᵢ at each bin i in the CDW, combined with features from RCᵢ, DPᵢ and perc_{chr}_{ₖ}, which is calculated independently from said chosen CDW;
(iii) choosing levels of significant α for RCᵢ and DPᵢ by comparing RCᵢ and/or DPᵢ at each bin at step (ii) to the mean of all RCᵢ and/or DPᵢ in the CDW;
(iv) one by one choosing any set of features from the plurality of quantitative signals including RCᵢ, DPᵢ, relative differences of RCᵢ and/or DPᵢ compared to mean RCᵢ and/or DPᵢ across all bins of the CDW, and perc_{chr}_{ₖ} for the learning model;
(v) one by one applying any learning model on the training set, with three main parameters including the size of CDW at step (i), the level of significance α or binaries encoding for RCᵢ and/or DPᵢ determined at step (iii), and the set of features chosen from quantitative signals at step (iv), and with the hyperparameters of the learning model defined during the training process;
wherein the learning model is selected from the group consisting of machine learning-based models, Gaussian Mixture Model (GMM), and Hidden Markov Model;
wherein machine learning-based models include Naïve Bayes, K-Nearest Neighbors, Random Forest, Multi-Layer Perceptron, Support Vector Machines, or any other known machine learning-based models;
(vi) applying the trained model at step (v) to predict the CNV on the test set and evaluating if the model achieves the targeted performance metrics;
wherein the targeted performance metrics include:
area under the curve (AUC) is from 0.9 and above;
accuracy is from 0.9 and above;
sensitivity is from 0.9 and above;
specificity is from 0.95 and above;
positive predictive value is from 0.75 and above; and
mean squared error (MSE) is below 0.2;
(vii) Rank the models that satisfy the targeted performance metrics and choose the best performance model as the final model for the CNV of interest; if no model is found to satisfy the targeted performance metrics, steps (iii) to (vii), or steps (iv) to (vii), or steps (v) to (vii), or steps (i) to (vii) will be repeated until a model is found; and
(f) applying the final model to predict CNVs, including microdeletion syndromes, or microduplications, or aneuploidies, or the number of sex chromosomes, using at least three parameters, including the size of CDW, the level of significance α or binaries encoding for RCᵢ and/or DPᵢ, and the set of features chosen from quantitative signals;
wherein chromosomal aneuploidies including Down syndrome (trisomy 21 or T21), Edward syndrome (trisomy 18 or T18), and Patau syndrome (trisomy 13 or T13);
wherein the microdeletion syndromes include DiGeorge syndrome, Wolf-Hirschhorn syndrome, Cri-du-chat syndrome, and Prader-Willis/Angelman syndrome;
wherein the sex chromosomes include chromosome X and chromosome Y.

2. The method of claim 1, wherein step (c) the average sequencing coverage (average sequencing) depth can vary from 0.1× to 1.2×.

3. The method of claim 1, wherein step (c) further includes samples with the obtained cfDNA sequencing data violate one or several quality control parameters, in which the total number of reads is of at least 18M paired-end reads, the average sequencing coverage or average sequencing depth is depth below 0.1×, the total number of reads successfully mapped relative to the total number of reads sequenced is less than 70%, and the read duplication rate is more than 30%.

4. The method of claim 1, wherein the method further includes training data for machine learning models generated by subsampling reads to simulate new biological samples;
wherein subsampling reads from real biological samples must ensure a limited percentage of overlapping reads between any two simulated samples is 10%, or 20%, or 30%, or 40%, or 50%, or 60%, or 70%, or 80%, or 90% of the total number of reads in each newly simulated sample; and
wherein a number of positive and negative new data points needed for machine learning-based models is selected one from the groups including from 50, from 100, from 150, from 200, from 250, from 300.

5. The method of claim 1, wherein the method further includes training data for machine learning models generated by data point simulation using the distribution estimated by the Gaussian Mixture Model (GMM);
wherein a new data point simulation by GMM-based distribution estimation is done from 10, from 20, from 30, from 40, and from 50 original data points; and
wherein a number of positive and negative new data points needed for machine learning-based models is selected one from the groups including from 50, from 100, from 150, from 200, from 250, from 300.

6. The method of claim 1, wherein step (e) the final model for CNV detection is machine learning-based models.

7. The method of claim 6, wherein said machine learning-based models for detecting genetic abnormality is Support Vector Machines.

8. The method of claim 6, wherein said machine learning-based models for sex chromosome detection is Multi-Layer Perceptron regression model.

9. The method of claim 1, wherein the CDW is selected from the regions consisting of one or more of chromosome 22q11.2 region (corresponding to DiGeorge syndrome), chromosome 15q11-q13 region between positions 22-28 Mb (corresponding to Prader Willi/Angelman syndrome), chromosome 4q12 region and/or chromosome 4p16.3 region (corresponding to Wolf-Hirschhorn syndrome), and chromosome 5p15.2-p15.33 region (corresponding to Cri-du-chat syndrome), whole chromosome 13 (corresponding to T13), whole chromosome 18 (corresponding to T18), whole chromosome 21 (corresponding to T21), whole chromosome X (for estimating the number of X), and whole chromosome Y (for estimating the number of Y).

10. The method of claim 9, wherein the chromosome 22q11.2 region comprises of one or more loci that represent the markers located in each of the inter-LCR22A-B region, the inter-LCR22A-C region, the inter-LCR22A-D region, the inter-LCR22B-D region, and the inter-LCR22C-D region;
the inter-LCR22A-B region is located between positions 19,000,000-20,500,000 bp on chromosome 22, including at least one marker from the list consisting of: PRODH, SLC25A1, CDC45, GP1BB, TBX1, TANGO2, and a combination thereof;
the inter-LCR22A-C region is located between positions 19,000,000-21,000,000 bp on chromosome 22, including at least one marker from the list consisting of: PRODH, SLC25A1, CDC45, GP1BB, TBX1, TANGO2, SCARF2, and a combination thereof;
the inter-LCR22A-D region is located between positions 19,000,000-21,500,500 bp on chromosome 22, including at least one marker from the list consisting of: PRODH, SLC25A1, CDC45, GP1BB, TBX1, TANGO2, SCARF2, SNAP29, LZTR1, and a combination thereof;
the inter-LCR22B-D region is located between positions 20,500,000-21,500,000 bp on chromosome 22, including at least one marker from the list consisting of: SCARF2, SNAP29, LZTR1, and a combination thereof;
the inter-LCR22C-D region is located between positions 21,000,000-21,500,000 bp on chromosome 22, including at least one marker from the list consisting of SNAP29, LZTR1, and a combination thereof.

11. The method of claim 9, wherein the 5p15.2-p15.33 region between positions 0.15-11.41 Mb on chromosome 5, including at least one marker selected from the list consisting of: CEP72, TPPP, TERT, SLC6A3, MRPL36, NDUFS6, MED10, ADCY2, MTRR, SEMASA, CCT5, CTNND2, and a combination thereof.

12. The method of claim 1, wherein the final model for DiGeorge syndrome is a Support Vector Machines model trained with the set of features comprising the relative count (RCᵢ), and the differences between RCᵢ and mean RC of all bins in the CDW encoded in binaries (0 and 1) or encoded in levels of significant difference α.

13. The method of claim 12, wherein the final model for DiGeorge syndrome is a Support Vector Machines model trained with the set of features chosen from the differences between RCᵢ and mean RC of all bins in the CDW encoded in the level of significant difference α.

14. The method of claim 1, wherein the final model for aneuploidies is a Support Vector Machines model trained with the set of features including the relative counts (RCᵢ), the differences between RCᵢ and mean RC of all bins in the CDW encoded in levels of significant difference α, the percentage of read counts of chromosomes of interest in the sample of interest over the total number of read counts in the sample of interest (per_{chr}_{ₖ}), and the difference in proportion (DPᵢ).

15. The method of claim 14, wherein the final model for aneuploidies is a Support Vector Machines model trained with the set of features including the percentage of read counts of chromosomes of interest in the sample of interest over the total number of read counts in the sample of interest perc_{chr}_{ₖ}, and the difference in proportion (DPᵢ).

16. The method of claim 1, wherein the final model for number of sex chromosome detection is a Multi-Layer Perceptron model trained with the set of features including the relative counts (RCᵢ), the differences between RCᵢ and mean RC of all bins in the CDW encoded in levels of significant difference α, the percentage of read counts of chromosomes of interest in the sample of interest over the total number of read counts in the sample of interest perc_{chr}_{ₖ}, and the difference in proportion (DPᵢ).

17. The method of claim 16, wherein the final model for number of sex chromosome detection is a Multi-Layer Perceptron model trained with the set of features including the percentage of read counts of chromosomes of interest in the sample of interest over the total number of read counts in the sample of interest perc_{chr}_{ₖ}, and the difference in proportion (DPᵢ).

18. The method of claim 1, wherein the method further comprises principal component analysis (PCA) correction to remove unwanted variations between fetal CNV-free samples.

19. The method of claim 18, wherein the principal component analysis (PCA) correction step to remove unwanted variations applied to aneuploidies detection.

20. A system for evaluation of fetal copy number variation (CNV) in a test sample through non-invasive prenatal testing (NIPT), the system comprising:
a sequencer for receiving cell-free deoxyribonucleic acid fragments (cfDNA fragments) from the test sample and providing cfDNA sequencing data of the test sample;
a computer; and
one or more computer-readable storage media having stored thereon instructions for execution on said computer to:
(a) collecting blood samples from pregnant women from 9 weeks of pregnancy;
(b) extracting cell-free deoxyribonucleic acid fragments (cfDNA fragments) from the blood samples, performing whole genome sequencing (WGS) on said extracted cfDNA fragments to obtain cfDNA sequencing data, and preprocessing cfDNA sequencing data by removing adapters, aligning and mapping reads to a referenced human genome;
(c) performing quality control on obtained cfDNA sequencing data for being accepted for the prediction based on the total number of reads, the total number of reads successfully mapped relative to the total number of reads sequenced, the average sequencing coverage or average sequencing depth, and the read duplication rate;
in which the total number of reads is of at least 18M paired-end reads;
the total number of reads successfully mapped relative to the total number of reads sequenced is at least 70%;
the average sequencing coverage or average sequencing depth is between 0.1× and 5×; and
the read duplication rate is below 30%;
(d) dividing a referenced genome into a plurality of non-overlapping bins and filtering the bins based on a predetermined GC-content threshold for bins;
wherein the bins on said referenced genome have size ranging from 50-5000 Kb;
wherein bins having GC-content between 30 and 70 are kept, and bins having GC-content below 30 or over 70 are removed;
(e) defining a CNV detection window (CDW), a bin size, a set of features for machine learning/deep learning models and fine-tune said model for detecting fetal CNV for selecting a final model, comprising the steps of:
(Step 1) Defining a plurality of CDWs, wherein a CDW is defined for each CNV type or subtype of interest, wherein a CDW encompasses a region on the genome where the CNV of interest is located, wherein a CDW is the region of CNV of interest, or of at least 50% bigger than the region of CNV of interest, in which bins are discarded unless located within the defined CDW;
(Step 2) Preparing a set of features for model learning by transforming the raw read counts of kept bins at (step 1) into a set of quantitative copy number signals at bins of interest, then using these quantitative signals as features for a model, allowing the model to learn from the features to detect CNV;
in which the features is selected from the set of quantitative signals (QS), including (A) relative counts (RCᵢ with i among the plurality of bins), (B) difference in proportion (DPᵢ with i among the plurality of bins), (C) relative differences of RCᵢ or DPᵢ and the mean RC or DP across all bins in the window of detection, and (D) percentages of read counts of chromosomes of interest in the sample of interest over the total number of read counts in the sample of interest perc_{chr}_{ₖ};
(A) RCᵢ is defined as:
$$R {C}_{i} = \frac{fetal read {counts}_{i}}{total read counts of sample}$$
(B) DPᵢ is defined as:
$$D {P}_{i} = \frac{fetal read {counts}_{i}}{total fetal read counts of sample} - \frac{maternal read {counts}_{i}}{total maternal read counts of sample}$$
wherein the fetal read counts correspond to the fetal fragment size, in which the fetal fragment size is below 140 base-pairs, below 145 base-pairs, below 150 base-pairs, below 155 base-pairs, below 160 base-pairs, below 160 base-pairs, or below 170 base-pairs;
wherein the maternal read counts correspond to the maternal fragment size, in which the maternal fragment size is above 160 base-pairs, above 165 base-pairs, above 170 base-pairs, and above 175 base-pairs;
(C) the relative difference between the quantitative signal RCᵢ or DPᵢ and the mean RC or DP of all bins across the CDW encoded in binaries (0 and 1) or encoded in levels of significant difference α;
wherein the relative difference between the quantitative signal RCᵢ or DPᵢ and the mean RC or DP of all bins across the CDW encoded in binaries (0 and 1);
$${QS}_{i} = \{ \begin{matrix}1 ( deletion ) if Q {S}_{i} & \leq mean ( {\sum }_{i = 1}^{n} {QS}_{i} ) - α \times \sqrt{\frac{{\sum }_{i = 1}^{n} {( {QS}_{i} - mean ( {\sum }_{i = 1}^{n} QS ) )}^{2}}{n}} \\ 1 ( duplication ) if Q {S}_{i} & \geq mean ( {\sum }_{i = 1}^{n} {QS}_{i} ) + α \times \sqrt{\frac{{\sum }_{i = 1}^{n} {( {QS}_{i} - mean ( {\sum }_{i = 1}^{n} QS ) )}^{2}}{n}} \\ 0 ( normal ) , otherwise & \end{matrix}$$
QSᵢ is the quantitative signal RCᵢ or DPᵢ at bin i;
α ranging between 0.5-2 indicates the significance of the relative difference between QSᵢ and mean QS of all bins across the CDW; and
n is the total number of bins of interest in the CDW;
wherein the relative difference between the quantitative signal RCᵢ or DPᵢ and the mean RC or DP of all bins across the CDW encoded in encoded in levels of significant difference α:
$${QS}_{i}^{′} = \{ \begin{matrix}α + b ( deletion ) if Q {S}_{i} & \leq mean ( {\sum }_{i = 1}^{n} {QS}_{i} ) - α \times \sqrt{\frac{{\sum }_{i = 1}^{n} {( {QS}_{i} - mean ( {\sum }_{i = 1}^{n} QS ) )}^{2}}{n}} \\ α + b ( duplication ) if Q {S}_{i} & \geq mean ( {\sum }_{i = 1}^{n} {QS}_{i} ) + α \times \sqrt{\frac{{\sum }_{i = 1}^{n} {( {QS}_{i} - mean ( {\sum }_{i = 1}^{n} QS ) )}^{2}}{n}} \\ 0 ( normal ) , otherwise & \end{matrix}$$
QSᵢ is the quantitative signal RCᵢ or DPᵢ at bin i;
QS′ᵢ is the new quantitative signal obtained from QSᵢ;
α ranging between 0.5-2 indicates the significance of the relative difference between QSᵢ and mean QS of all bins across the CDW, b=0.5 if α ranging between 0.5 and 1.5,
b=1 if α ranging between 0 and 2; and
n is the total number of bins of interest in the CDW;
(D) perc_{chr}_{ₖ} is defined as:
$$p {erc}_{c h {r}_{k}} = \frac{R P {M}_{c h {r}_{k}}}{Total RPM} \times 100 with {RPM}_{c h {r}_{k}} = \frac{\frac{read {counts}_{c h {r}_{k}}}{{I}_{c h {r}_{k}}}}{{\sum }_{j = 1}^{23} \frac{read {counts}_{c h {r}_{k}}}{{I}_{c h {r}_{k}}}}$$
l is the length of chromosome of interest;
RPM_{chr}_{ₖ} is the normalized read counts of chromosome of interest in the sample of interest; and
Total RPM is the total normalized number of read counts in the sample of interest;
(Step 3) fine-tuning models for detecting fetal CNV by repeating the following steps until a set of parameters is determined for a specific CNV;
(i) one by one through each defined CDW for each CNV at (step 1), for which one can apply a strategy of sliding window of at least 1 Mb with seeding length of four consecutive bins;
(ii) calculating the relative differences of RCᵢ and DPᵢ at each bin i in the CDW combined with features from RCᵢ, DPᵢ and perc_{chr}_{ₖ}, which is calculated independently from said chosen CDW;
(iii) choosing levels of significant α for RCᵢ and DPᵢ by comparing RCᵢ and/or DPᵢ at each bin at step (ii) to the mean of all RCᵢ and/or DPᵢ in the CDW;
(iv) one by one choosing any set of features from the plurality of quantitative signals including RCᵢ, DPᵢ, relative differences of RCᵢ and/or DPᵢ compared to mean RCᵢ and/or DPᵢ across all bins of the CDW, and perc_{chr}_{ₖ} for the learning model;
(v) one by one applying any learning model on the training set, with three main parameters including the size of CDW at step (i), the level of significance α or binaries encoding for RCᵢ and/or DPᵢ determined at step (iii), and the set of features chosen from quantitative signals at step (iv), and with the hyperparameters of the learning model defined during the training process;
wherein the learning model is selected from the group consisting of machine learning-based models, Gaussian Mixture Model (GMM), and Hidden Markov Model;
wherein machine learning-based models include Naïve Bayes, K-Nearest Neighbors, Random Forest, Multi-Layer Perceptron, Support Vector Machines, or any other known machine learning-based models;
(vi) applying the trained model at step (v) to predict the CNV on the test set and evaluating if the model achieves the targeted performance metrics;
wherein the targeted performance metrics include:
area under the curve (AUC) is from 0.9 and above;
accuracy is from 0.9 and above;
sensitivity is from 0.9 and above;
specificity is from 0.95 and above; and
positive predictive value is from 0.75 and above;
mean squared error (MSE) is below 0.2;
(vii) Rank the models that satisfy the targeted performance metrics and choose the best performance model as the final model for the CNV of interest; if no model is found to satisfy the targeted performance metrics, steps (iii) to (vii), or steps (iv) to (vii), or steps (v) to (vii), or steps (i) to (vii) will be repeated until a model is found; and
(f) applying the final model to predict CNVs, including microdeletion syndromes, or microduplications, or aneuploidies, or the number of sex chromosomes, using at least three parameters, including the size of CDW, the level of significance α or binaries encoding for RCᵢ and/or DPᵢ, and the set of features chosen from quantitative signals;
wherein chromosomal aneuploidies including Down syndrome (trisomy 21 or T21), Edward syndrome (trisomy 18 or T18), and Patau syndrome (trisomy 13 or T13);
wherein the microdeletion syndromes include DiGeorge syndrome, Wolf-Hirschhorn syndrome, Cri-du-chat syndrome, and Prader-Willis/Angelman syndrome;
wherein the sex chromosomes include chromosome X and chromosome Y.
